(19) United States
(12) Patent Application Publication
(10) Pub. No.: US 2025/0319611 A1
(43) Pub. Date: Oct. 16, 2025

(54) VESSEL SEALER END EFFECTOR

(71) Applicant: Virtual Incision Corporation, Lincoln, NE (US)

(72) Inventors: Derek Eilers, Denver, CO (US); Shane Farritor, Lincoln, NE (US); Nathan Wood, Lincoln, NE (US)

(21) Appl. No.: 19/177,261

(22) Filed: Apr. 11, 2025

Related U.S. Application Data
(60) Provisional application No. 63/632,554, filed on Apr. 11, 2024.

(51) Int. Cl. B25J 15/00; A61B 34/30; B25J 15/02; B25J 19/00

(52) U.S. Cl. CPC B25J 15/0023; A61B 34/30; B25J 15/0226; B25J 19/0025; A61B 2560/04

(57) ABSTRACT

Various vessel sealer end effectors comprising at least one actuation mechanism having a rotatable drive rod, a linear drive body operably coupled to the rotatable drive rod, and an operational component such as a jaw or deployable blade operably coupled to the linear drive body. Some end effector embodiments have two such actuation mechanisms, with one of the rotatable drive rods being coaxial with and disposed at least partially within the other of the rotatable drive rods. Other implementations include one such actuation mechanism operably coupled to one or more jaws and another actuation mechanism that is a linear actuation mechanism operably coupled to a deployable cutting blade. Still other implementations have two rotatable jaws that rotate around the same center of rotation such that the jaws can operate with a wrist-like action.

### CROSS-REFERENCE TO RELATED APPLICATION(S)

This application claims the benefit under 35 U.S.C. § 119(e) to U.S. Provisional Application 63/632,554, filed Apr. 11, 2024 and entitled “Vessel Sealer End Effector,” which is hereby incorporated herein by reference in its entirety.

### FIELD

The various embodiments herein relate to surgical devices (and, in some embodiments, to robotic surgical devices) having end effectors for use in various surgical procedures. In some versions, the end effectors are releasable from the surgical devices and interchangeable with other end effectors.

### BACKGROUND

Invasive surgical procedures are essential for addressing various medical conditions. When possible, minimally invasive procedures, such as laparoscopy, are preferred. However, known minimally invasive technologies such as laparoscopy are limited in scope and complexity due in part to the need to remove and insert new surgical tools into the body cavity when changing surgical instruments due to the size of the access ports. Known robotic systems such as the da Vinci® Surgical System (available from Intuitive Surgical, Inc., located in Sunnyvale, CA) are also restricted by the access ports and trocars, the necessity for medical professionals to remove and insert new surgical tools into the abdominal cavity, as well as having the additional disadvantages of being very large, very expensive, unavailable in most hospitals, and having limited sensory and mobility capabilities.

Various robotic surgical tools have been developed to perform certain procedures inside a target cavity of a patient. These robotic systems are intended to replace the standard laparoscopic tools and procedures—such as, for example, the da Vinci@ system—that involve the insertion of long surgical tools through trocars positioned through incisions in the patient such that the surgical tools extend into the target cavity and allow the surgeon to perform a procedure using the long tools. As these systems are developed, various new components are developed to further improve the operation and effectiveness of these systems.

There is a need in the art for an improved vessel sealer end effector for use with medical devices, including robotic surgical devices and systems.

### BRIEF SUMMARY

Discussed herein are various end effectors that can be coupled to various types of medical devices, including various non-robotic or robotic surgical devices and systems.

In Example 1, a vessel sealer end effector comprises an end effector body comprising a body lumen defined through the end effector body and a rotatable jaw joint disposed at or near a distal end of the end effector body. The end effector further comprises a rotatable jaws drive screw rotatably disposed within the body lumen, the rotatable jaws drive screw comprising a proximal screw coupling structure disposed at a proximal end of the rotatable jaws drive screw, screw threads disposed around an outer surface of the rotatable jaws drive screw at a distal end of the rotatable jaws drive screw, and a drive screw lumen defined within the rotatable jaws drive screw. In addition, the end effector comprises first and second jaws rotatably coupled to the end effector body at the rotatable jaw joint, wherein each of the first and second jaws comprise a proximal slot defined in a proximal body of each of the first and second jaws and a jaws linear drive shaft comprising a proximal tubular body comprising a linear drive lumen comprising drive threads disposed on an inner surface of the linear drive lumen, wherein the distal end of the rotatable jaws drive screw is positionable within the linear drive lumen such that the drive threads are mateable with the screw threads, distal prongs disposed at a distal end of the jaws linear drive shaft, and a drive rod disposed between the distal prongs such that the drive rod is slidably disposed within the proximal slots defined in the first and second jaws. Further, the end effector comprises a deployable blade slidably disposed through the end effector body, wherein the deployable blade is movable between a retracted position within the end effector body and a deployed position between the first and second jaws.

Example 2 relates to the vessel sealer end effector according to Example 1, wherein each of the first and second jaws further comprises a structural backbone extending along a length of the jaw, wherein the proximal body extends proximally from the structural backbone, a first insulation layer disposed around a first portion of the structural backbone, a second insulation layer disposed around a second portion of the structural backbone, a contact surface disposed over the first insulation layer and attached to the second insulation layer, and a blade track formed along a length of the contact surface and the first insulation layer.

Example 3 relates to the vessel sealer end effector according to Example 1, wherein the deployable blade comprises an elongate proximal rod slidably disposed within the drive screw lumen, a distal blade body attached to the elongate proximal rod, and a blade slot defined along a length of the distal blade body, wherein the drive rod is slidably disposed within the blade slot.

Example 4 relates to the vessel sealer end effector according to Example 3, further comprising an actuation shaft slidably disposed within the drive screw lumen such that the actuation shaft is slidable into contact with a proximal end of the elongate proximal rod and a tensioned component operably coupled to the deployable blade, wherein the tensioned component is in an untensioned state when the deployable blade is in the retracted position.

Example 5 relates to the vessel sealer end effector according to Example 1, wherein the deployable blade comprises a distal blade body, a blade linear drive shaft disposed near a proximal end of the distal blade body, and a blade slot defined along a length of the distal blade body, wherein the drive rod is slidably disposed within the blade slot.

Example 6 relates to the vessel sealer end effector according to Example 5, wherein the blade linear drive shaft further comprises a linear drive lumen comprising drive threads disposed on an inner surface of the linear drive lumen.

Example 7 relates to the vessel sealer end effector according to Example 6, further comprising a rotatable blade drive screw rotatably disposed at least partially within the drive screw lumen, the rotatable jaws drive screw comprising a proximal screw coupling structure disposed at a proximal end of the rotatable blade drive screw and screw threads disposed around an outer surface of the rotatable blade drive screw at a distal portion of the rotatable blade drive screw, wherein a distal portion of the rotatable blade drive screw is positionable within the linear drive lumen of the blade linear drive shaft such that the drive threads of the linear drive lumen are mateable with the screw threads of the rotatable jaws drive screw.

In Example 8, a vessel sealer end effector comprises an end effector body comprising a body lumen defined through the end effector body and a rotatable jaw joint disposed at or near a distal end of the end effector body. In addition, the end effector comprises a rotatable drive screw rotatably disposed within the body lumen, the rotatable drive screw comprising a proximal screw coupling structure disposed at a proximal end of the rotatable drive screw, screw threads disposed around an outer surface of the rotatable drive screw at a distal end of the rotatable drive screw, and a drive screw lumen defined within the rotatable drive screw. The end effector also comprises first and second jaws rotatably coupled to the end effector body at the rotatable jaw joint, wherein each of the first and second jaws comprise a proximal slot defined in a proximal body of each of the first and second jaws, and a linear drive shaft comprising a proximal tubular body comprising a linear drive lumen comprising drive threads disposed on an inner surface of the linear drive lumen, wherein the distal end of the rotatable drive screw is positionable within the linear drive lumen such that the drive threads are mateable with the screw threads, a hub disposed at a distal end of the linear drive shaft, and drive tabs disposed on the hub such that the drive tabs are slidably disposed within the proximal slots defined in the first and second jaws. Further, the end effector comprises a deployable blade slidably disposed through the end effector body, wherein the deployable blade is movable between a retracted position within the end effector body and a deployed position between the first and second jaws.

Example 9 relates to the vessel sealer end effector according to Example 8, wherein the hub comprises two channels defined in an outer surface of the hub, wherein the two channels are sized and shaped to receive elongate wires electrically coupled to the first and second jaws.

In Example 10 a vessel sealer end effector comprises an end effector body comprising a body lumen defined through the end effector body and a first rotatable drive rod rotatably disposed within the body lumen, the first rotatable drive rod comprising a first proximal rod coupling structure disposed at a proximal end of the first rotatable drive rod, first rod threads disposed around an outer surface of the first rotatable drive rod along a distal portion of the first rotatable drive rod, and a first drive rod lumen defined within the first rotatable drive rod. The end effector also comprises a first linear drive body comprising a first linear drive lumen defined within the first linear drive body and first drive threads disposed on an inner surface of the first linear drive lumen, wherein a distal portion of the first rotatable drive rod is position able within the first linear drive lumen such that the first drive threads are mateable with the first rod threads. In addition, the end effector comprises a first connecting link operably coupled to the first linear drive body and a second rotatable drive rod rotatably disposed at least partially within the first drive rod lumen, the second rotatable drive rod comprising a second proximal rod coupling structure disposed at a proximal end of the second rotatable drive rod and second rod threads disposed around an outer surface of the second rotatable drive rod along a distal portion of the second rotatable drive rod. Further, the end effector comprises a second linear drive body comprising a second linear drive lumen defined within the second linear drive body and second drive threads disposed on an inner surface of the second linear drive lumen, wherein a distal portion of the second rotatable drive rod is positionable within the second linear drive lumen such that the second drive threads are mateable with the second rod threads. The end effector also comprises a second connecting link operably coupled to the second linear drive body.

Example 11 relates to the vessel sealer end effector according to Example 10, further comprising a first operational component operably coupled to the first connecting link and a second operational component operably coupled to the second connecting link.

Example 12 relates to the vessel sealer end effector according to Example 11, wherein the first operational component comprises a first jaw and the second operational component comprises a second jaw.

Example 13 relates to the vessel sealer end effector according to Example 11, wherein the first operational component comprises at least one jaw and the second operational component comprises a deployable blade.

Example 14 relates to the vessel sealer end effector according to Example 13, wherein the at least one jaw comprises a rotatable first jaw comprising a proximal body, wherein the proximal body comprises a slot defined therein and a stationary second jaw, and wherein the first connecting link comprises a pin, wherein the pin is slidably disposed within the slot, wherein linear movement of the first connecting link causes rotation of the rotatable first jaw.

Example 15 relates to the vessel sealer end effector according to Example 13, wherein the at least one jaw comprises first and second rotatable jaws, wherein each of the first and second rotatable jaws comprises a proximal body, wherein the proximal body comprises a slot defined therein, and wherein the first connecting link comprises a pin, wherein the pin is slidably disposed within the slots of the first and second rotatable jaws, wherein linear movement of the first connecting link causes rotation of the first and second jaws.

Example 16 relates to the vessel sealer end effector according to Example 10, further comprising a first jaw rotatably coupled to the first connecting link at a first center of rotation, and a second jaw rotatably coupled to the second connecting link at a second center of rotation, wherein the first and second jaws can rotate independently of each other.

Example 17 relates to the vessel sealer end effector according to Example 16, wherein the first and second centers of rotation are coaxial.

Example 18 relates to the vessel sealer end effector according to Example 17, wherein the first and second jaws are configurated to be positioned in a closed configuration, wherein the first and second jaws are not parallel with a longitudinal axis of the end effector body.

Example 19 relates to the vessel sealer end effector according to Example 18, wherein the first and second jaws are configured to move from the closed configuration into an open configuration.

Example 20 relates to the vessel sealer end effector according to Example 10, wherein the second proximal rod coupling structure is disposed proximally of the first proximal rod coupling structure.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a view of a robotic surgical system in an operating room or other surgical space, according to one embodiment.

FIG. 2 is a perspective view of robotic surgical device, according to one embodiment.

FIG. 3A is a side view of a forearm of a surgical device with a surgical end effector attached thereto, according to one embodiment.

FIG. 3B is a perspective view of the body of the forearm of FIG. 3A, according to one embodiment.

FIG. 3C is a side view of some of the components of the forearm and end effector of FIG. 3A, according to one embodiment.

FIG. 3D is a side view of some other components of the forearm and end effector of FIG. 3A, according to one embodiment.

FIG. 3E is a side view of a proximal portion of the end effector of FIG. 3A, according to one embodiment.

FIG. 3F is a side view of the proximal portion of the end effector of FIG. 3E with a portion of the end effector body removed, according to one embodiment.

FIG. 3G is another side of the proximal portion of the end effector of FIG. 3E with a portion of the end effector body removed and other components visible, according to one embodiment.

FIG. 4A is a side view of a drive screw of an end effector, according to one embodiment.

FIG. 4B is a side view of a contact lead of an end effector, according to one embodiment.

FIG. 4C is a side view of a portion of an end effector body, according to one embodiment.

FIG. 4D is a perspective view of a linear drive component of an end effector body, according to one embodiment.

FIG. 4E is a perspective view of a drive pin that can be used in combination with a linear drive component of an end effector body, according to one embodiment.

FIG. 5 is a side view of end effector jaws, according to one embodiment.

FIG. 6A is a perspective view of one of the end effector jaws of FIG. 5, according to one embodiment.

FIG. 6B is a cross-sectional view of the end effector jaw of FIG. 6A, according to one embodiment.

FIG. 6C is a perspective view of the contact plate of the end effector jaw of FIG. 6A, according to one embodiment.

FIG. 6D is a perspective view of the insulation layer of the end effector jaw of FIG. 6A, according to one embodiment.

FIG. 6E is a perspective view of the structural support of the end effector jaw of FIG. 6A, according to one embodiment.

FIG. 6F is a perspective view of the base layer of the end effector jaw of FIG. 6A, according to one embodiment.

FIG. 7A is a perspective view of a contact plate with gap control features, according to another embodiment.

FIG. 7B is a perspective view of a contact plate without gap control features, according to another embodiment.

FIG. 8A is a side view of some of the components of a forearm and end effector, according to one embodiment.

FIG. 8B is a side cross-sectional view of the jaws and some internal components of the end effector of FIG. 8A, according to one embodiment.

FIG. 9 is a side view of the cutting blade of the end effector of FIG. 8A, according to one embodiment.

FIG. 10 is a side, cross-sectional view of a proximal portion of the end effector of FIG. 8A, according to one embodiment.

FIG. 11 is a perspective view of a proximal portion of the end effector of FIG. 8A, according to one embodiment.

FIG. 12A is a side view of a surgical end effector, according to another embodiment.

FIG. 12B is a cross-sectional top view of certain components of the end effector of FIG. 12A, according to one embodiment.

FIG. 12C is a perspective view of a distal portion of the end effector of FIG. 12A, according to one embodiment.

FIG. 12D is a front view of the distal end of the end effector of FIG. 12A, according to one embodiment.

FIG. 12E is a cross-sectional side view of the hub of the end effector of FIG. 12A, according to one embodiment.

FIG. 12F is a cross-sectional front view of the hub of the end effector of FIG. 12A, according to one embodiment.

FIG. 12G is a perspective view of one jaw of the end effector of FIG. 12A, according to one embodiment.

FIG. 12H is a close-up perspective view of a proximal portion of the jaw of FIG. 12G, according to one embodiment.

FIG. 12I is a top view of certain components of the end effector of FIG. 12A, according to one embodiment.

FIG. 12J is a perspective view of certain components of the end effector of FIG. 12A, according to one embodiment.

FIG. 13A is a top view of a distal portion of an end effector jaw, according to one embodiment.

FIG. 13B is a side view of the distal portion of the end effector jaw of FIG. 13A, according to one embodiment.

FIG. 14A is a top view of a distal portion of another end effector jaw, according to a further embodiment.

FIG. 14B is a side view of the distal portion of the end effector jaw of FIG. 14A, according to one embodiment.

FIG. 15A is a top view of a distal portion of a further end effector jaw, according to yet another embodiment.

FIG. 15B is a side view of the distal portion of the end effector jaw of FIG. 15A, according to one embodiment.

FIG. 16A is a cross-sectional side view of a surgical end effector, according to another embodiment.

FIG. 16B is a cross-sectional side view of a proximal portion of the end effector of FIG. 16A, according to one embodiment.

FIG. 16C is a perspective view of certain components of the end effector of FIG. 16A, according to one embodiment.

FIG. 17A is a cross-sectional side view of a distal portion of a surgical end effector in which the jaw is in a closed or near-closed configuration, according to yet another embodiment.

FIG. 17B is a cross-sectional side view of the end effector of FIG. 17A in which the jaw is in the open configuration, according to one embodiment.

FIG. 18A is a cross-sectional side view of a surgical end effector in which the jaws are in a closed configuration, according to a further embodiment.

FIG. 18B is a cross-sectional side view of the end effector of FIG. 18A in which the jaws are in the open configuration, according to one embodiment.

FIG. 19A is a schematic side view of certain internal components of another surgical end effector, according to a further embodiment.

FIG. 19B is a schematic perspective view of the distal portion of the surgical end effector of FIG. 19A, according to one embodiment.

FIG. 19C is a schematic side view of the distal portion of the surgical end effector of FIG. 19A in which the jaws are in the closed configuration, according to one embodiment.

FIG. 19D is a schematic side view of the distal portion of the surgical end effector of FIG. 19A in which the jaws are in a rotated and closed configuration, according to one embodiment.

FIG. 19E is a schematic side view of the distal portion of the surgical end effector of FIG. 19A in which the jaws are in a rotated and open configuration, according to one embodiment.

FIG. 20A is a schematic side view of certain internal components of another surgical end effector, according to a further embodiment.

FIG. 20B is a schematic side view of certain internal components of the surgical end effector of FIG. 20A in which the jaws are in an open configuration, according to one embodiment.

FIG. 20C is a schematic side view of certain internal components of the surgical end effector of FIG. 20A in which the jaws are in a rotated and near-closed configuration, according to one embodiment.

FIG. 20D is a schematic side view of certain internal components of the surgical end effector of FIG. 20A in which the jaws are in a rotated and open configuration, according to one embodiment.

FIG. 20E is another schematic side view of certain internal components of the surgical end effector of FIG. 20D with additional measurements depicted, according to one embodiment.

### DETAILED DESCRIPTION

The various systems and devices disclosed herein relate to devices for use in medical procedures and systems. More specifically, various embodiments relate to vessel sealer end effectors and forearms with such vessel sealer end effectors for integration into or use with the various robotic devices and related methods and systems.

It is understood that the various embodiments of vessel sealer end effectors and forearms and related methods and systems disclosed herein can be incorporated into or used with any other known medical devices, systems, and methods.

For example, the various embodiments disclosed herein may be incorporated into or used with any of the medical devices and systems disclosed in U.S. Pat. No. 8,968,332 (issued on Mar. 3, 2015 and entitled “Magnetically Coupleable Robotic Devices and Related Methods”), U.S. Pat. No. 8,834,488 (issued on Sep. 16, 2014 and entitled “Magnetically Coupleable Surgical Robotic Devices and Related Methods”), U.S. Pat. No. 10,307,199 (issued on Jun. 4, 2019 and entitled “Robotic Surgical Devices and Related Methods”), U.S. Pat. No. 9,579,088 (issued on Feb. 28, 2017 and entitled “Methods, Systems, and Devices for Surgical Visualization and Device Manipulation”), U.S. Patent Application 61/030,588 (filed on Feb. 22, 2008), U.S. Pat. No. 8,343,171 (issued on Jan. 1, 2013 and entitled “Methods and Systems of Actuation in Robotic Devices”), U.S. Pat. No. 8,828,024 (issued on Sep. 9, 2014 and entitled “Methods and Systems of Actuation in Robotic Devices”), U.S. Pat. No. 9,956,043 (issued on May 1, 2018 and entitled “Methods and Systems of Actuation in Robotic Devices”), U.S. patent application Ser. No. 15/966,606 (filed on Apr. 30, 2018 and entitled “Methods, Systems, and Devices for Surgical Access and Procedures”), U.S. patent application Ser. No. 12/192,663 (filed on Aug. 15, 2008 and entitled “Medical Inflation, Attachment, and Delivery Devices and Related Methods”), U.S. patent application Ser. No. 15/018,530 (filed on Feb. 8, 2016 and entitled “Medical Inflation, Attachment, and Delivery Devices and Related Methods”), U.S. Pat. No. 8,974,440 (issued on Mar. 10, 2015 and entitled “Modular and Cooperative Medical Devices and Related Systems and Methods”), U.S. Pat. No. 8,679,096 (issued on Mar. 25, 2014 and entitled “Multifunctional Operational Component for Robotic Devices”), U.S. Pat. No. 9,179,981 (issued on Nov. 10, 2015 and entitled “Multifunctional Operational Component for Robotic Devices”), U.S. Pat. No. 9,883,911 (issued on Feb. 6, 2018 and entitled “Multifunctional Operational Component for Robotic Devices”), U.S. patent application Ser. No. 15/888,723 (filed on Feb. 5, 2018 and entitled “Multifunctional Operational Component for Robotic Devices”), U.S. Pat. No. 8,894,633 (issued on Nov. 25, 2014 and entitled “Modular and Cooperative Medical Devices and Related Systems and Methods”), U.S. Pat. No. 8,968,267 (issued on Mar. 3, 2015 and entitled “Methods and Systems for Handling or Delivering Materials for Natural Orifice Surgery”), U.S. Pat. No. 9,060,781 (issued on Jun. 23, 2015 and entitled “Methods, Systems, and Devices Relating to Surgical End Effectors”), U.S. Pat. No. 9,757,187 (issued on Sep. 12, 2017 and entitled “Methods, Systems, and Devices Relating to Surgical End Effectors”), U.S. Pat. No. 10,350,000 (issued on Jul. 16, 2019 and entitled “Methods, systems, and devices relating to surgical end effectors”), U.S. patent application Ser. No. 16/512,510 (filed on Jul. 16, 2019 and entitled “Methods, Systems, and Devices Relating to Surgical End Effectors”), U.S. Pat. No. 9,089,353 (issued on Jul. 28, 2015 and entitled “Robotic Surgical Devices, Systems, and Related Methods”), U.S. Pat. No. 10,111,711 (issued on Oct. 30, 2018 and entitled “Robotic Surgical Devices, Systems, and Related Methods”), U.S. patent application Ser. No. 16/123,619 (filed on Sep. 6, 2018 and entitled “Robotic Surgical Devices, Systems and Related Methods”), U.S. Pat. No. 9,770,305 (issued on Sep. 26, 2017 and entitled “Robotic Surgical Devices, Systems, and Related Methods”), U.S. patent application Ser. No. 15/661,147 (filed on Jul. 27, 2017 and entitled “Robotic Devices with On Board Control & Related Systems & Devices”), U.S. patent application Ser. No. 13/833,605 (filed on Mar. 15, 2013 and entitled “Robotic Surgical Devices, Systems, and Related Methods”), U.S. patent application Ser. No. 13/738,706 (filed on Jan. 10, 2013 and entitled “Methods, Systems, and Devices for Surgical Access and Insertion”), U.S. patent application Ser. No. 14/661,465 (filed on Mar. 18, 2015 and entitled “Methods, Systems, and Devices for Surgical Access and Insertion”), U.S. patent application Ser. No. 15/890,860 (filed on Feb. 7, 2018 and entitled “Methods, Systems, and Devices for Surgical Access and Insertion”), U.S. Pat. No. 9,498,292 (issued on Nov. 22, 2016 and entitled “Single Site Robotic Devices and Related Systems and Methods”), U.S. Pat. No. 10,219,870 (issued on Mar. 5, 2019 and entitled “Single site robotic device and related systems and methods”), U.S. Patent Application 16/293, 135 (filed Mar. 3, 2019 and entitled “Single Site Robotic Device and Related Systems and Methods”), U.S. Pat. No. 9,010,214 (issued on Apr. 21, 2015 and entitled “Local Control Robotic Surgical Devices and Related Methods”), U.S. Pat. No. 10,470,828 (issued on Nov. 12, 2019 and entitled “Local Control Robotic Surgical Devices and Related Methods”), U.S. patent application Ser. No. 16/596,034 (filed on Oct. 8, 2019 and entitled “Local Control Robotic Surgical Devices and Related Methods”), U.S. Pat. No. 9,743,987 (issued on Aug. 29, 2017 and entitled “Methods, Systems, and Devices Relating to Robotic Surgical Devices, End Effectors, and Controllers”), U.S. patent application Ser. No. 15/687,787 (filed on Aug. 28, 2017 and entitled “Methods, Systems, and Devices Relating to Robotic Surgical Devices, End Effectors, and Controllers”), U.S. Pat. No. 9,888,966 (issued on Feb. 13, 2018 and entitled “Methods, Systems, and Devices Relating to Force Control Surgical Systems”), U.S. patent application Ser. No. 15/894,489 (filed on Feb. 12, 2018 and entitled “Methods, Systems, and Devices Relating to Force Control Surgical Systems”), U.S. patent application Ser. No. 14/212,686 (filed on Mar. 14, 2014 and entitled “Robotic Surgical Devices, Systems, and Related Methods”), U.S. patent application Ser. No. 14/334,383 (filed on Jul. 17, 2014 and entitled “Robotic Surgical Devices, Systems, and Related Methods”), U.S. patent application Ser. No. 14/853,477 (filed on Sep. 14, 2015 and entitled “Quick-Release End Effectors and Related Systems and Methods”), U.S. patent application Ser. No. 16/504,793 (filed on Jul. 8, 2019 and entitled “Quick-Release End Effectors and Related Systems and Methods”), U.S. Pat. No. 10,376,322 (issued on Aug. 13, 2019 and entitled “Robotic Device with Compact Joint Design and Related Systems and Methods”), U.S. patent application Ser. No. 16/538,902 (filed on Aug. 13, 2019 and entitled “Robotic Device with Compact Joint Design and Related Systems and Methods”), U.S. patent application Ser. No. 15/227,813 (filed on Aug. 3, 2016 and entitled Robotic Surgical Devices, System and Related Methods”) U.S. patent application Ser. No. 15/599,231 (filed on May 18, 2017 and entitled “Robotic Surgical Devices, Systems, and Related Methods”), U.S. patent application Ser. No. 15/687,113 (filed on Aug. 25, 2017 and entitled “Quick-Release End Effector Tool Interface”), U.S. patent application Ser. No. 15/691,087 (filed on Aug. 30, 2017 and entitled “Robotic Device with Compact Joint Design and an AdditionalDegree of Freedom and Related Systems and Methods”), U.S. Patent Application 15/821, 169 (filed on Nov. 22, 2017 and entitled “Gross Positioning Device and Related Systems and Methods”), U.S. Patent Application 15/826, 166 (filed on Nov. 29, 2017 and entitled “User controller with user presence detection and related systems and methods”), U.S. patent application Ser. No. 15/842,230 (filed on Dec. 14, 2017 and entitled “Releasable Attachment Device for Coupling to Medical Devices and Related Systems and Methods”), U.S. patent application Ser. No. 16/144,807 (filed on Sep. 27, 2018 and entitled “Robotic Surgical Devices with Tracking Camera Technology and Related Systems and Methods”), U.S. patent application Ser. No. 16/241,263 (filed on Jan. 7, 2019 and entitled “Single-Manipulator Robotic Device With Compact Joint Design and Related Systems and Methods”), U.S. Pat. No. 7,492,116 (filed on Oct. 31, 2007 and entitled “Robot for Surgical Applications”), U.S. Pat. No. 7,772,796 (filed on Apr. 3, 2007 and entitled “Robot for Surgical Applications”), and U.S. Pat. No. 8,179,073 (issued on May 15, 2011, and entitled “Robotic Devices with Agent Delivery Components and Related Methods”), all of which are hereby incorporated herein by reference in their entireties.

Certain device and system implementations disclosed in the applications listed above can be positioned within a body cavity of a patient, or a portion of the device can be placed within the body cavity, in combination with a support component similar to those disclosed herein. An “in vivo device” as used herein means any device that can be positioned, operated, or controlled at least in part by a user while being positioned within a body cavity of a patient such that the entire device is disposed within the body cavity or the device is disposed through an orifice or incision such that a distal portion of the device is disposed within the body cavity while a proximal portion is disposed outside of the patient's body. Further, an “in vivo device” can include any device that is coupled to a support component such as a rod or other such component that is disposed through an opening or orifice of the body cavity, also including any device positioned substantially against or adjacent to a wall of a body cavity of a patient, further including any such device that is internally actuated (having no external source of motive force), and additionally including any device that may be used laparoscopically or endoscopically during a surgical procedure. As used herein, the terms “robot,” and “robotic device” shall refer to any device that can perform a task either automatically or in response to a command.

Certain embodiments provide for insertion of the present invention into the cavity while maintaining sufficient insufflation of the cavity. Further embodiments minimize the physical contact of the surgeon or surgical users with the present invention during the insertion process. Other implementations enhance the safety of the insertion process for the patient and the present invention. For example, some embodiments provide visualization of the present invention as it is being inserted into the patient's cavity to ensure that no damaging contact occurs between the system/device and the patient. In addition, certain embodiments allow for minimization of the incision size/length. Other implementations include devices that can be inserted into the body via an incision or a natural orifice. Further implementations reduce the complexity of the access/insertion procedure and/or the steps required for the procedure. Other embodiments relate to devices that have minimal profiles, minimal size, or are generally minimal in function and appearance to enhance ease of handling and use.

As in manual laparoscopic procedures, a known insufflation system can be used to pump sterile carbon dioxide (or other gas) into the patient's abdominal cavity. This lifts the abdominal wall from the organs and creates space for the robot. In certain implementations, the system has no direct interface with the insufflation system. Alternatively, the system can have a direct interface to the insufflation system.

In certain implementations in which the device is inserted through an insertion port, the insertion port is a known, commercially-available flexible membrane placed transabdominally to seal and protect the abdominal incision. This off-the-shelf component is the same device or substantially the same device that is used in substantially the same way for Hand-Assisted Laparoscopic Surgery (HALS). The only difference is that the arms of the robotic device according to the various embodiments herein are inserted into the abdominal cavity through the insertion port rather than the surgeon's hand. The robotic device body seals against the insertion port when it is positioned therethrough, thereby maintaining insufflation pressure. The port is single-use and disposable. Alternatively, any known port can be used. In further alternatives, the device can be inserted through an incision without a port or through a natural orifice.

Certain implementations disclosed herein relate to “combination” or “modular” medical devices that can be assembled in a variety of configurations. For purposes of this application, both “combination device” and “modular device” shall mean any medical device having modular or interchangeable components that can be arranged in a variety of different configurations.

Certain embodiments disclosed or contemplated herein can be used for colon resection, a surgical procedure performed to treat patients with lower gastrointestinal diseases such as diverticulitis, Crohn's disease, inflammatory bowel disease and colon cancer. Approximately two-thirds of known colon resection procedures are performed via a completely open surgical procedure involving an 8- to 12-inch incision and up to six weeks of recovery time. Because of the complicated nature of the procedure, existing robot-assisted surgical devices are not always used for colon resection surgeries, and manual laparoscopic approaches are only used in one-third of cases. In contrast, the various implementations disclosed herein can be used in a minimally invasive approach to a variety of procedures that are typically performed ‘open’ by known technologies, with the potential to improve clinical outcomes and health care costs. Further, the various implementations disclosed herein can be used for any laparoscopic surgical procedure in place of the known mainframe-like laparoscopic surgical robots that reach into the body from outside the patient. That is, the less-invasive robotic systems, methods, and devices disclosed herein feature small, self-contained surgical devices that are inserted in their entireties through a single incision in the patient's abdomen. Designed to utilize existing tools and techniques familiar to surgeons, the devices disclosed herein will not require a dedicated operating room or specialized infrastructure, and, because of their much smaller size, are expected to be significantly less expensive than existing robotic alternatives for laparoscopic surgery. Due to these technological advances, the various embodiments herein could enable a minimally invasive approach to procedures performed in open surgery today.

FIG. 1 depicts one embodiment of a robotic surgical system 10 with which any of the forearm and/or vessel sealer end effector embodiments disclosed or contemplated herein can be used. The components of the various system implementations can include an external control console 16 and a robotic device 12 having a removable camera 14 as will also be described in additional detail below. In accordance with the implementation of FIG. 1, the robotic device 12 is shown mounted to the operating table 18 via a known, commercially available support arm 20. The system 10 can be, in certain implementations, operated by the surgeon 22 at the console 16 and one surgical assistant 24 positioned at the operating table 18. Alternatively, one surgeon 22 can operate the entire system 10. In a further alternative, three or more people can be involved in the operation of the system 10. It is further understood that the surgeon (or user) 22 can be located at a remote location in relation to the operating table 18 such that the surgeon 22 can be in a different city or country or on a different continent from the patient on the operating table 18.

In this specific implementation, the robotic device 12 with the camera 14 are both connected to the surgeon console 16 via cables: a device cable 24A and a camera cable 24B that will be described in additional detail below. Alternatively, any connection configuration can be used. In certain implementations, the system can also interact with other devices during use such as a electrosurgical generator, an insertion port, and auxiliary monitors.

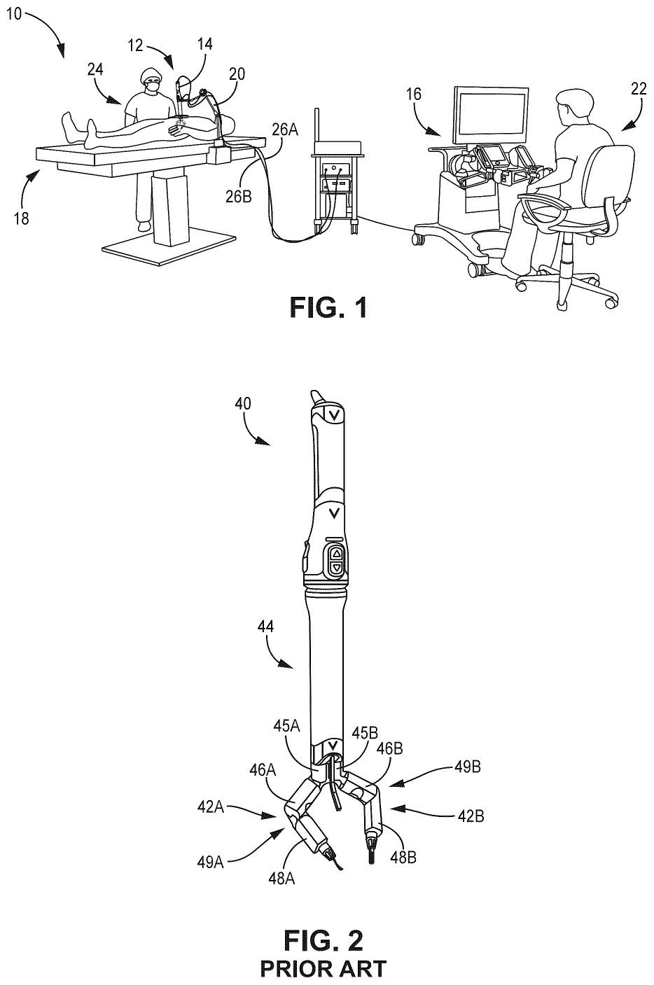

FIG. 2 depicts one exemplary implementation of a robotic device 40 that can be incorporated into the exemplary system 10 discussed above or any other system disclosed or contemplated herein. Any of the forearm and/or vessel sealer end effector implementations disclosed or contemplated herein can be added to one or both of the arms 42A, 42B of the device 40. In certain implementations, the arms 42A, 42B can have first segments (or upper arms) 46A, 46B coupled to the device body 44 at shoulders (or shoulder joints) 45A, 45B and second segments (or forearms) 48A, 48B coupled to the upper arms 46A, 46B at elbows (or elbow joints) 49A, 49B. Various versions of the device 40 are disclosed in additional detail in various of the patents/applications incorporated by reference above.

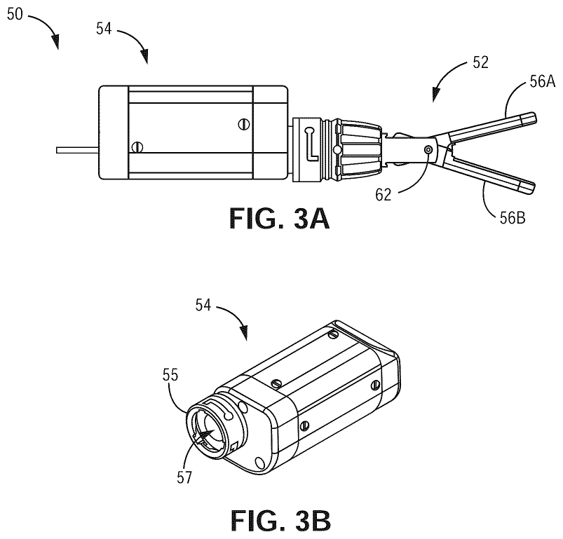

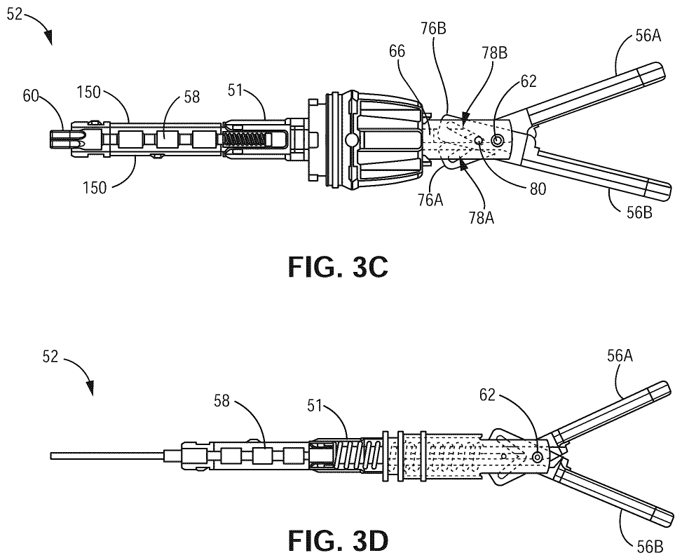

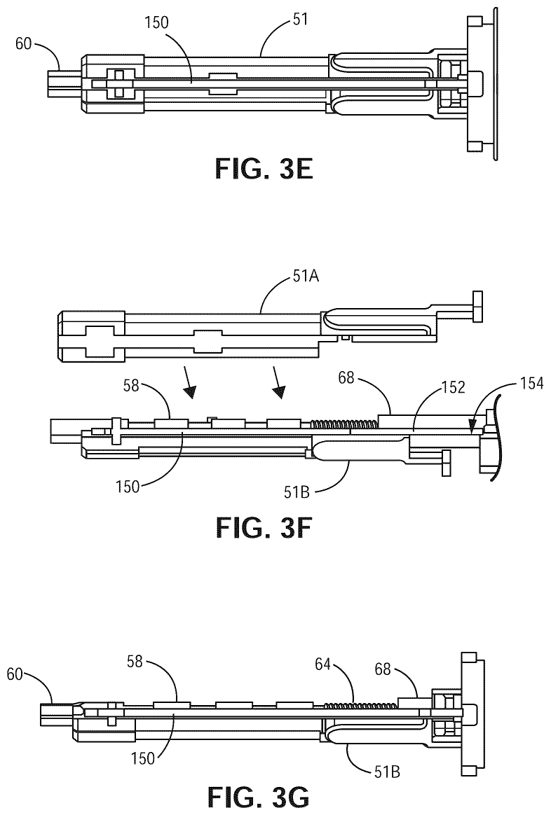

One specific implementation of a forearm 50 with a vessel sealing end effector 52 coupled thereto as depicted in FIG. 3A. More specifically, FIG. 3A depicts a forearm 50 having a forearm body 54, with the end effector 52 having jaws 56A, 56B removably coupled to the body 54. FIG. 3B depicts the forearm body 54, including the coupling collar 55 and the lumen 57 into which the various vessel sealer end effectors (or other types of end effectors) as disclosed herein (such as end effector 52, for example) can be positioned. Further, various images of the end effector 52 are shown in additional detail in FIGS. 3B-3F.

In various embodiments, the exemplary forearm 50 (and any other forearm disclosed or contemplated herein to which any of the various end effector implementations herein can be coupled) can be substantially similar to the forearm embodiments disclosed in U.S. patent application Ser. No. 16/736,329, which was filed on Jan. 7, 2020 and is entitled “Robotically Assisted Surgical System and Related Devices and Methods;” U.S. patent application Ser. No. 17/367,915, which was filed on Jul. 6, 2021 and is entitled “Robotic Surgical Devices with Tracking Camera Technology and Related Systems and Methods;” or U.S. patent application Ser. No. 18/317,175, which was filed on May 15, 2023 and is entitled “Robotic Surgical Devices, Systems, and Related Methods,” all of which are hereby incorporated herein by reference in their entireties. Further, any of the various end effector embodiments disclosed or contemplated herein can be coupled to any forearm or any other robotic arm as disclosed or contemplated in any of the various patents and applications incorporated by reference herein or any other known forearm or robotic arm.

In accordance with certain implementations, the forearm 50 and the end effector 52 are configured such that the end effector 52 can be quickly attached to and removed from the forearm 50. In fact, any of the various forearm and end effector embodiments disclosed or contemplated herein can have substantially the same quick-release coupling components, mechanisms, and features disclosed in U.S. patent application Ser. No. 18/167,953, which was filed on Feb. 13, 2023 and is entitled “Quick-Release End Effectors and Related Systems and Methods,” which is hereby incorporated herein by reference in its entirety. Alternatively, the various end effector and forearm implementations disclosed or contemplated herein can incorporate any known quick-release components, mechanisms, or features.

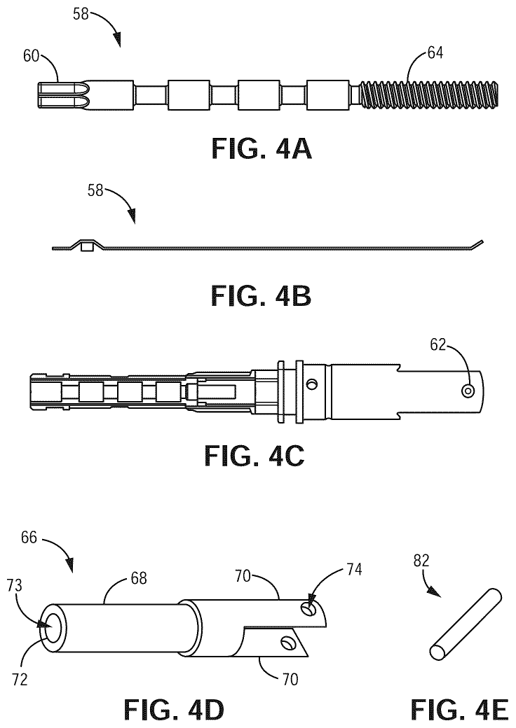

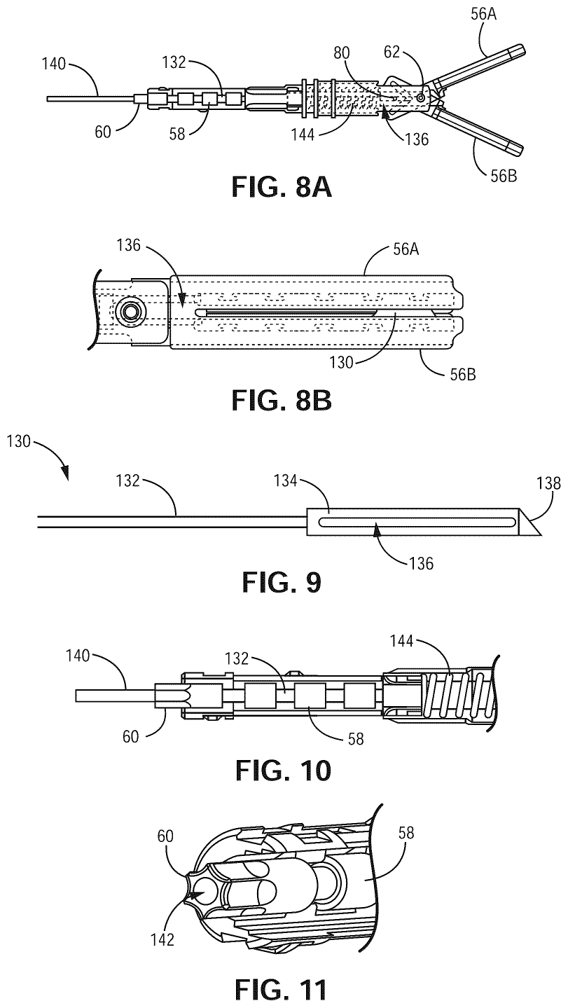

In this exemplary embodiment, the end effector 52 has both (1) an actuation assembly for urging the jaws 56A, 56B between their open and closed positions, as shown in FIGS. 3C-4E, and (2) a blade actuation assembly for urging the cutting blade between its retracted and deployed positions, as shown in FIGS. 8A-11.

Turning first to the actuation assembly for urging the jaws 56A, 56B between their open and closed positions, FIGS. 3C and 3D (as well as FIGS. 3E-G, and 4A) depict the jaw actuation assembly, including a drive or lead screw 58 that is rotatably disposed through the end effector body (or “shell”) 51. According to one embodiment, the end effector body 51 has two coupleable sections 51A, 51B as shown in FIGS. 3F and 3G. Alternatively, the body 51 can be a single unitary component or can be made up of three or more coupleable sections. As best shown in FIGS. 3C, 3G, 4A, and 11, the drive screw 58 has a coupling feature or component 60 at its proximal end that is operably coupleable to a drive motor (not shown) within the forearm 50. More specifically, in one embodiment, the coupling component 60 is a torx component 60, but alternatively can be any known component for removable coupling to a drive component and/or motor. In use, a drive component (such as a drive shaft or drive gear) (not shown) in the forearm 50 can be operably coupled to the coupling component 60 such that the drive shaft or gear can drive the rotation of the drive screw 58.

As best shown in FIGS. 3C, 3F-G, 4A, and 4D, the drive screw 58 is operably coupled to the jaws 56A, 56B such that rotation of the screw 58 causes rotation of each jaw 56A, 56B (between the open and closed positions) around a jaw joint 62 (as shown in FIGS. 3A-D). More specifically, as best shown in FIGS. 3F-G and 4A, the drive screw 58 has threads 64 defined or otherwise disposed at or near the distal end of the screw 58 that mateably couple with the threads (not shown) defined on the inner surface of the lumen 73 in the linear drive component (or “clevis”) 66 (as shown in FIGS. 3C and 4D). As best shown in FIG. 4D, the linear drive component 66 has a proximal tube 68 with a lumen 73 defined therethrough that is accessible via a proximal opening 72. Further, the drive component 66 also has distal members (or “prongs”) 70 with an opening 74 defined through a distal portion thereof. As best shown in FIGS. 3F and 3G, the threads 64 of the drive screw 58 are positioned through the opening 72 in the proximal end of the proximal tube 68 of the linear drive component 66 and mate with the threads (not shown) on the inner surface of the lumen 73 therein such that rotation of the drive screw 58 causes linear movement of the linear drive component 66. Rotation of the screw 58 in one direction causes the linear drive component 66 to move distally along the longitudinal axes of the screw 58 and drive component 66 (which are generally or substantially coaxial), while rotation of the screw 58 in the other direction causes the drive component 66 to move proximally along the same axis.

As best shown in FIGS. 3C and 4D, the linear drive component 66 is operably coupled to the jaws 56A, 56B via the distal prongs 70 such that linear movement of the drive component 66 along the longitudinal axis of the drive component 66 causes the jaws 56A, 56B to move between their open and closed positions. More specifically, each of the jaws 56A, 56B has a proximal body 76A, 76B with a slot 78A, 78B defined therein. Further, as shown in FIG. 3C a drive pin 80 (similar to pin 82 as shown in FIG. 4E) can be disposed through the opening 74 in the linear drive component 66 and further be positioned within the slots 78A, 78B of the proximal bodies 76A, 76B of the jaws 56A, 56B. As such, linear movement of the linear drive component 66 causes the drive pin 80 to move linearly along the axis of the drive component 66 within the slots 78A, 78B, thereby causing the jaws 56A, 56B to rotate around the axis 62 between the open and closed positions. It is noted that the configuration of the jaw joint 62 can include a pin 62 (that can be substantially similar to pin 82 as shown in FIG. 4E) such that the jaws 56A, 56B rotate around the pin 62 that creates the axis 62 as discussed above.

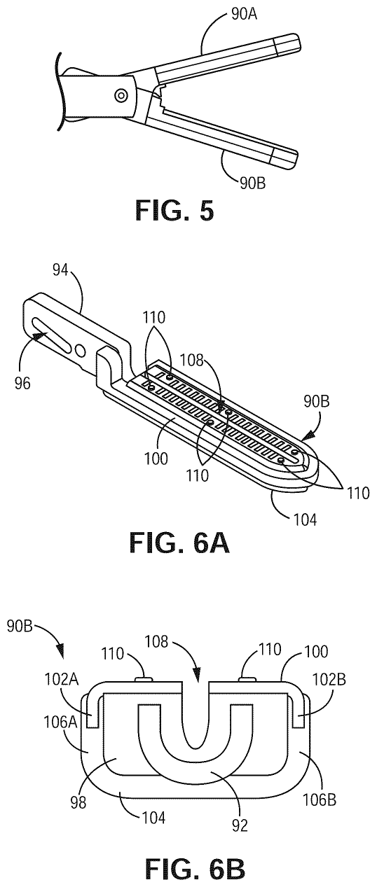

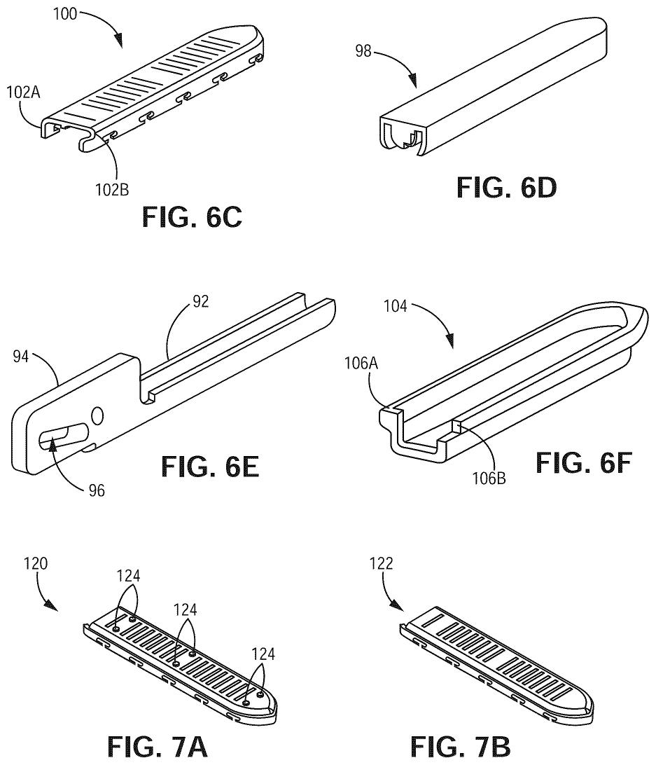

One implementation of a pair of jaws 90A, 90B that can be incorporated into any of the vessel sealer end effector embodiments herein is depicted in additional detail in FIGS. 5-7B. The two separate jaws 90A, 90B are shown in FIG. 5, while FIGS. 6A-6F depict the various components of the lower jaw 90B, but it is understood that upper jaw 90A is substantially similar to the lower jaw 90B such that the description of the lower jaw 90B and the components therein can also apply to the upper jaw 90A. In other words, both of the jaws 90A, 90B in accordance with certain embodiments can have the structure as shown in FIGS. 6A-6F. As shown in FIGS. 6A, 6B, and 6E, the jaw 90 has a structural support or “backbone” 92 which extends along the length of the jaw 90, with a proximal body 94 extending proximally therefrom with a slot 96 defined therein (wherein the proximal body 94 and slot 96 can be substantially similar to the proximal bodies 76A-B and slots 78A-B discussed above). In one implementation, the backbone 92 can be made of stainless steel or any other known metal with similar rigidity.

For clarity, FIG. 6B is a cross-sectional view of the lower jaw 90B. The cross-sectional depiction allows one to clearly see the backbone 92 discussed above, along with an insulation layer 98, a contact plate 100 that forms the contact surface (with sides 102A, 102B), a base layer 104 (with sides 106A, 106B), a knife track 108 to receive a deployable blade, and gap control features (bumps) 110, all of which are discussed in detail below.

In addition, as shown in FIGS. 6B and 6D, the jaw 90 also has an insulation layer 98 disposed over the backbone 92 that can be positioned within and around the backbone 92 as shown. According to certain embodiments, the insulation layer 98 can be made of plastic and, in some implementations can be injection molded plastic that is molded over the backbone 92 such that the insulation layer 98 can insulate the backbone 92 from the contact plate 100 that is disposed over the insulation layer 98 to form the electrically conductive contact surface of the jaw 90. As shown in FIGS. 6A-C, the contact plate 100 can also have sides 102A, 102B that extend downward (transverse to the plane of the contact surface) to assist with attachment of the plate 100 to the rest of the jaw 90. In an exemplary embodiment, the plate 100 is be made of stainless steel and can have a thickness of from about 0.008 inches to about 0.010 inches. Alternatively, the plate 100 can be made of any known electrically conductive metal having characteristics similar to stainless steel that can serve as the contact surface of the jaw of a vessel sealer end effector.

As shown in FIGS. 6A, 6B, and 6F, the jaw 90 also has a base layer 104 that is positioned on the side of the jaw 90 opposite the contact plate 100 and has sides 106A, 106B that also constitute the sides 106A, 106B of the jaw 90. In one embodiment, the sides 106A, 106B are coupled to the sides 102A, 102B of the contact plate 100 such that the base layer 104 helps to fix or otherwise retain the contact plate 100 in place. In one exemplary embodiment, the base layer 104 is made of plastic and, in some implementations can be injection molded plastic that is molded over the sides 102A, 102B of the contact plate 100 to couple the contact plate 100 thereto.

As shown in FIGS. 6A and 6B, the jaw 90B also has a knife track 108 defined in the contact surface side of the jaw 90B. The knife track 108 extends along the length of the jaw 90B and is sized to receive a deployable blade (such as the cutting apparatus 130 as discussed below) such that a bottom portion of the blade (or, in the case of an upper jaw like jaw 90A, the top portion of the blade) can move distally and proximally within the track 108 during use. Hence the track 108 can help to maintain the position of the blade as it is urged along the track 108.

In certain embodiments, as best shown in FIGS. 6A and 6B, the jaw 90B can also have at least two protrusions or bumps 110 formed on the contact plate 100 as shown. The bumps 110 are gap control features 110 that are made of a non-conductive material such as a ceramic material. Alternatively, the bumps 110 can be made of any known non-conductive material that can serve the same purposes as a ceramic material. The bumps 110 in one implementation have a height ranging from about 0.002 inches to about 0.006 inches. In certain implementations, the gap control features 110 are included on the contact plate 100 of only one of the two jaws in a pair.

For example, the two exemplary contact plates 120, 122 shown in FIGS. 7A and 7B, according to certain implementations, are two contact plates 120, 122 that could be attached to the two jaws 90A, 90B of FIG. 5 (and any pair of jaws according to any embodiment herein). In one specific embodiment, the contact plate 120 with the gap control features 124 (similar to the features 110 discussed above) is attached to the lower jaw 90B, while the contact plate 122 without the gap control features is attached to the upper jaw 90A. Alternatively, the plate 120 with the gap control features 124 is attached to the upper jaw 90A.

According to certain embodiments, the jaw 90 can also have a coating (not shown) disposed on the outer surfaces of the jaw 90. More specifically, the coating is a non-stick coating. In one embodiment, the coating is Teflon. Alternatively, the non-stick coating can be made of PTFE or ceramic material. In certain embodiments, any of these coating embodiments can be nano-coatings.

In certain exemplary implementations, the specific configuration of the jaw 90 as shown in FIGS. 5-7B can ensure delivery of the electrical energy to the active jaws (such as jaws 56A, 56B and/or jaws 90A, 90B as discussed elsewhere herein) in either monopolar or bipolar embodiments while preventing the jaws/device from shorting out. That is, as best shown in FIGS. 3B and 6D (and as discussed above), both the drive pin 80 and the joint pin 62 electrically couple the structural backbones 92 of the two jaws 56A, 56B. Thus, to prevent the jaws (such as jaws 56A, 56B and/or 90A, 90B) from being electrically connected through these contact points, the active part of each jaw (the contact plate, such as plate 100 as discussed above) is insulated from the structural backbone 92 of the jaw via the insulation layers 98, 104 as discussed above.

Further, the electrical energy is delivered separately to each of the contact plates 100 in each jaw (such as jaws 56A-B or 90A-B) via an insulated contact lead 150 (as best shown in FIGS. 3D-F, and 4B) that is electrically coupled to an insulated wire 152 (as best shown in FIG. 3E), which extends from the contact lead 150 to one of the two jaws 56A-B, 90A-B and is electrically coupled to the contact plate 100 therein. In one embodiment, each wire 152 passes through a separate channel 154 as shown in FIG. 3E to the target jaw.

One implementation of a blade actuation assembly or mechanism is depicted in additional detail in FIGS. 8A-11. The moveable cutting apparatus 130 as shown in FIG. 9 has an elongate proximal rod 132, an elongate distal blade body 134 with a slot 136 defined therethrough, and a distal blade 138. The blade body 134 has a narrow rectangular cross-sectional shape such that the blade body 134 and the blade 138 can be slidably disposed within the knife track 108 as discussed above. In one specific embodiment, the blade body 134 can have a thickness of about 0.006 inches and a height of about0.125 inches. Alternatively, the blade body 134 can have any dimensions that allow the blade body 134 to operate as described herein. Further, the slot 136 is defined within the body 134 along the length thereof as shown such that the joint pin 62 and the drive pin 80 can be disposed within the slot 136 (as shown in FIGS. 8A and 8B). As such, the slot 136 allows the cutting apparatus 130 to move linearly along the longitudinal axis of the apparatus 130 while the joint pin 62 and drive pin 80 are disposed within the slot 136 with the longitudinal axes of both pins 62, 80 being transverse to the longitudinal axis of the apparatus 130.

The cutting apparatus 130 can be actuated to move distally and proximally within the end effector in the following fashion, according to one embodiment. As shown in FIGS. 8A and 10, a flexible actuation shaft 140 is provided that extends into the proximal end of the drive screw 58 via a lumen 142 defined through the coupling feature 60 and the drive screw 58 (as best shown in FIG. 11). In one embodiment, the lumen 142 has a diameter of about 0.045 inches. Alternatively, the lumen 142 can have any diameter that allows for receiving the flexible shaft 140. The elongate proximal rod 132 is also disposed within the lumen 142 of the drive screw 58 such that the distal end of the flexible actuation shaft 140 can be urged distally into contact with a proximal end of the elongate proximal rod 132 (as best shown in FIG. 10). Thus, the elongate proximal rod 132 can be actuated by being contacted and urged distally by the actuation shaft 140. The actuation shaft 140 can be actuated at its proximal end via a known actuation device or mechanism that can be disposed either in the forearm or the upper arm to which the forearm is coupled. Thus, the actuation device or mechanism can cause the actuation shaft 140 to move distally within the lumen 142, thereby contacting the elongate proximal rod 132 and urging it distally. (The proximal rod 132 can be returned to its retracted position via a tensioning mechanism 144, as discussed in further detail below.) The known actuation device or mechanism can be a linear actuating mechanism, a motor/lead screw mechanism, a motor-driven cable mechanism with a rack and pinion, or any other known device or mechanism. According to one specific embodiment, the distance that the cutting apparatus 130 can move distally and proximally ranges from about 15 mm to about 18 mm. Alternatively, the cutting apparatus 130 can move any distance between its retracted position (as shown in FIG. 8A) and its deployed position (as shown in FIG. 8B).

In one implementation, the cutting apparatus 130 has a tensioning mechanism 144 (such as a spring 144) coupled thereto that urges the apparatus 130 into its retracted position as shown in FIG. 8A. That is, the retracted position of the cutting apparatus 130 is its untensioned position such that an external force is required to urge the cutting apparatus 130 into its deployed position (as shown in FIG. 8B). Thus, the only actuation or force required to cause the cutting apparatus 130 to move between its retracted and deployed positions is a distal force provided by the actuation shaft 140. In other words, the actuation shaft 140 can be urged distally by the known actuation device or mechanism at a sufficient force to overcome the tensioning mechanism 144 such that the distal end of the shaft 140 will urge the proximal end of the proximal rod 132 distally until the cutting apparatus 130 is disposed in its deployed position as shown in FIG. 8B. On the other hand, the cutting apparatus 130 can return to its retracted position simply by removing the force applied by the actuation shaft 140 such that the tensioning mechanism 144 urges the cutting apparatus 130 proximally until it is in the retracted position as shown in FIG. 8A. Thus, the actuation shaft 140 need not be coupled to the proximal rod 132 in order to be used in combination with the rod 132 to actuate the cutting apparatus 130 (urge the cutting apparatus 130 into its deployed position). Alternatively, the actuation shaft 140 and the proximal rod 132 can be coupled together such that the actuation shaft 140 can both push and pull the proximal rod 132 (and thus no tensioning mechanism is needed in such alternative embodiments).

According to one embodiment, the cutting apparatus 130 can be urged into its deployed position while the jaws 56A, 56B are closed as shown in FIG. 8B.

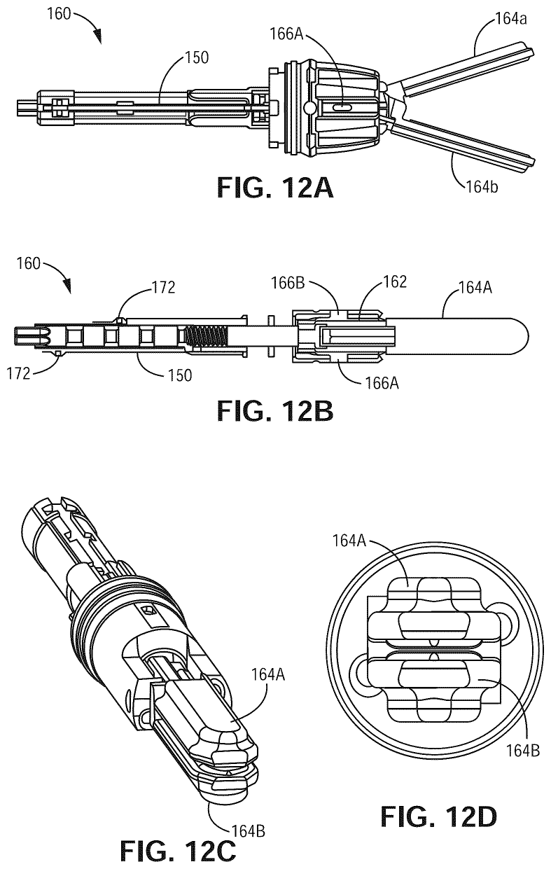

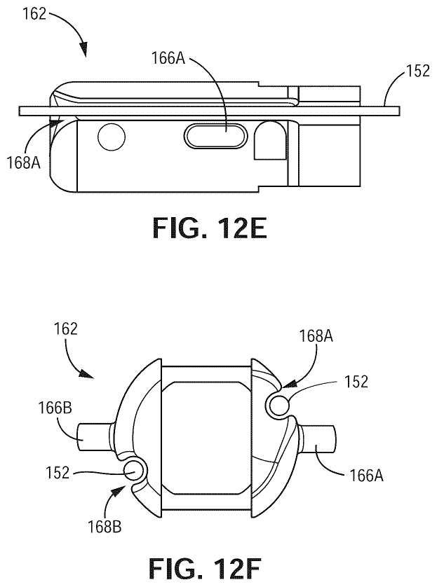

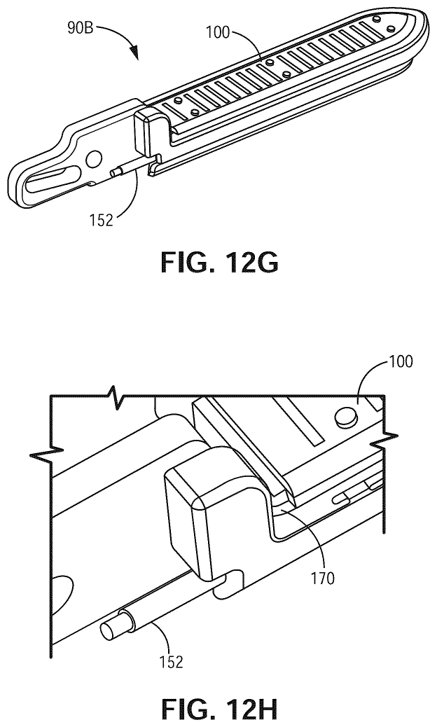

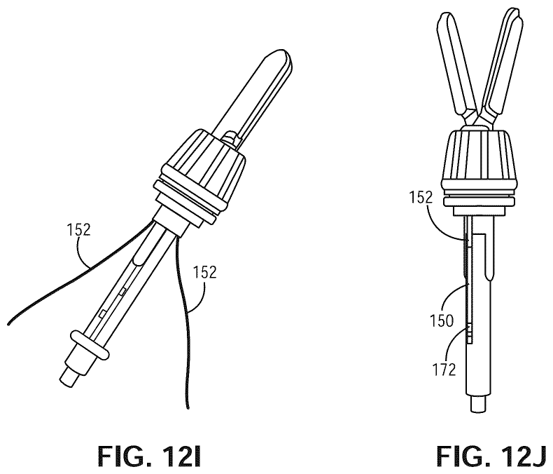

Another end effector embodiment 160 is depicted in FIGS. 12A-12J. According to certain versions, this end effector 160 has no deployable knife. In this implementation, the device 160 has a hub 162 that couples the jaws 164A, 164B to the proximal portion of the end effector 160 via tabs 166A, 166B that extend from the sides as best shown in FIGS. 12A-B and 12E-F. According to one embodiment, the hub 162 is made of a substantially rigid plastic or other non-conductive material. In the exemplary implementation as best shown in FIGS. 12E and 12F, the hub 162 has two grooves or channels 168A, 168B defined in the outer surface of the hub 162, each of which can receive one of the insulated wires 152 discussed above. In this embodiment, as best shown in FIGS. 12G and 12H, each of the insulated wires 152 is electrically coupled at its distal end to the contact plate 100 of one of the jaws (such as jaws 90A, 90B). While FIGS. 12G and 12H depict solely the jaw 90B, it is understood that a wire 152 is electrically coupled to the contact plate 100 of jaw 90A in a similar fashion. As best shown in FIG. 12H, the uninsulated portion of the wire 152 is electrically coupled to a tab or protrusion 170 extending from the proximal end of the contact plate 100. Alternatively, the wire 152 can be electrically coupled to the contact plate 100 in any known fashion and/or via any known component or mechanism. From the distal end, each wire 152 extends proximally through the relevant channel 168A, 168B of the hub 162 as shown in FIGS. 12E and 12F and extends out of the proximal end of the hub 162 as shown in FIGS. 12I and 12J. More specifically, FIG. 12I depicts one implementation of the end effector 160 during manufacture, in which the two insulated wires 152 extend proximally out of the hub 162 prior to the wires 152 being cut to a shorter length for attachment to the contact leads 150. As shown in FIG. 12J, once the wires 152 are cut, the proximal end of each insulated wire 152 is electrically coupled or soldered to a separate contact lead 150 (as discussed above with respect to FIGS. 3D-F, and 4B) such that the end effector 160 has two separate contact leads 150 disposed on opposite sides thereof. As shown in FIG. 12J (and also in FIGS. 12A and 12B), each lead 150 has a contact 172 protruding radially from the end effector 160 such that, when the end effector 160 is coupled to an arm of a robotic device (such as forearm 54 as discussed above), each contact 172 is in electrical contact with a contact ring (not shown) within the lumen of the arm (such as lumen 57 of forearm 54 as discussed above). Alternatively, the contacts 172 can be electrically coupled to corresponding contact rings in any forearm embodiment disclosed in U.S. patent application Ser. No. 18/167,953, which is incorporated by reference above. As such, the end effector 160 (and any other end effector embodiment herein) can be a quick-release end effector that can be used with any of the forearm embodiments herein or in U.S. patent application Ser. No. 18/167,953 as discussed above.

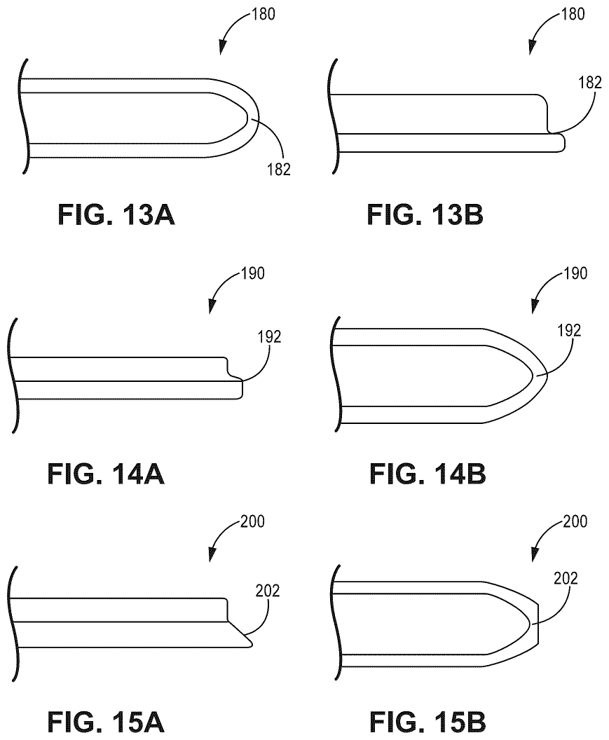

Any of the various vessel sealing end effector embodiments herein can also have jaws with specific configurations at their tips to facilitate tissue manipulation and/or dissection. More specifically, FIGS. 13A-15B depict certain optional jaw tip implementations. FIGS. 13A and 13B depict a jaw tip 180 with a beveled lip 182. Alternatively, FIGS. 14A and 14B depict a jaw tip 190 with a non-beveled lip 192. In a further alternative, FIGS. 15A and 15B depict a jaw tip 200 with a substantially flat-ended lip 202.

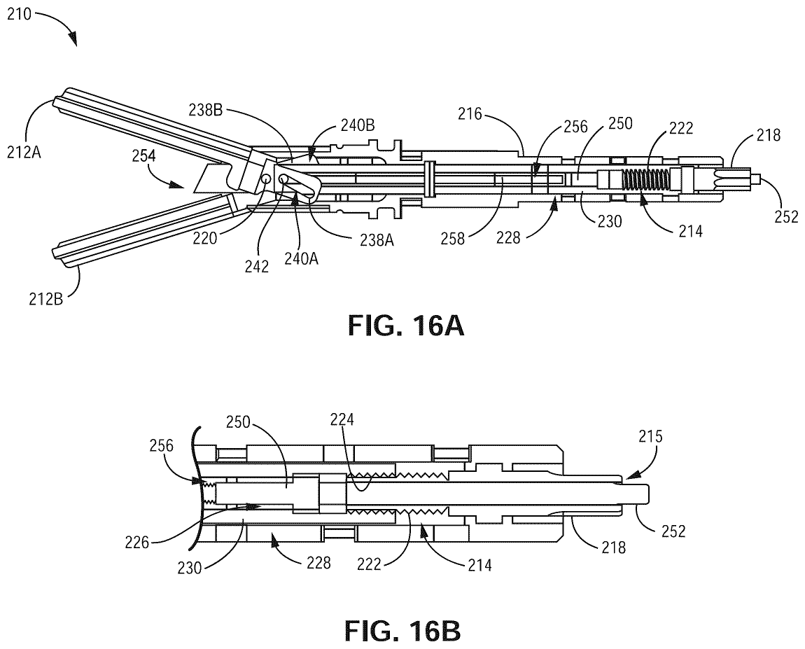

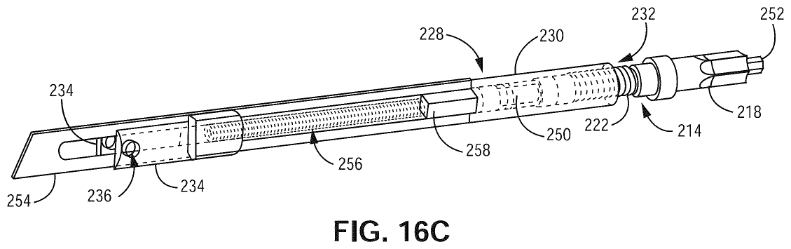

FIGS. 16A-16C depict yet another exemplary implementation of an end effector 210 having both (1) a jaw actuation assembly for urging the jaws 212A, 212B between their open and closed positions, and (2) a blade actuation assembly for urging the cutting blade between its retracted and deployed positions. As will be described in further detail below, the jaw actuation assembly includes a drive screw 214 rotatably coupled to a linear drive component 228 that is operably coupled to the jaws 212A, 212B such that rotation of the drive screw 214 causes axial or linear movement of the linear drive component 228, which causes rotation of the jaws 212A, 212B around their jaw joint 220. As will also be described in further detail below, the blade actuation assembly includes a drive screw 250 rotatably coupled to a linear drive component 258 that is fixed attached to or integral with the blade 254 such that rotation of the drive screw 250 causes axial or linear movement of the linear drive component 258 and the attached blade 254. Each actuation assembly will be described in additional detail below.

Turning first to the actuation assembly for urging the jaws 212A, 212B between their open and closed positions (the “jaw actuation assembly”), FIGS. 16A-C depict the jaw actuation assembly, including a drive or lead screw 214 that is rotatably disposed through the end effector body (or “shell”) 216. As best shown in FIG. 16A, like the body 51 as described above, in certain embodiments, the end effector body 216 can have two coupleable sections (not shown), can be made up of three or more coupleable sections, or can be a single unitary component as shown. As shown in FIGS. 16A-C, the drive screw 214 has a coupling feature or component 218 at its proximal end that is operably coupleable to a drive motor (not shown) within the forearm (not shown). More specifically, in one embodiment, the coupling component 218 is a torx component 218, but alternatively can be any known component for removable coupling to a drive component and/or motor. In use, a drive component (such as a drive shaft or drive gear) (not shown) in the forearm can be operably coupled to the coupling component 218 such that the drive shaft or gear can drive the rotation of the drive screw 214.

As best shown in FIGS. 16A and 16C, the drive screw 214 is operably coupled to the jaws 212A, 212B such that rotation of the screw 214 causes rotation of each jaw 212A, 212B (between the open and closed positions) around a jaw joint 220 (as shown in FIG. 16A). More specifically, as best shown in FIG. 16B, the drive screw 214 has threads 222 defined or otherwise disposed at or near the distal end of the screw 214 that mateably couple with the threads 224 defined on the inner surface of the lumen 226 in the linear drive component (or “clevis”) 228. Much like the linear drive component 66 as discussed above, as best shown in FIG. 16C, the linear drive component 228 has a proximal tube 230 with a lumen 226 defined therethrough that is accessible via a proximal opening 232. Further, as best shown in FIG. 16C, the drive component 228 also has distal members (or “prongs”) 234 with an opening 236 defined through a distal portion thereof. As also shown in FIG. 16C, the threads 222 of the drive screw 214 are positioned through the opening 232 in the proximal end of the proximal tube 230 of the linear drive component 228 and mate with the threads 224 on the inner surface of the lumen 226 therein such that rotation of the drive screw 214 causes linear movement of the linear drive component 228. Rotation of the screw 214 in one direction causes the linear drive component 228 to move distally along the longitudinal axes of the screw 214 and drive component 228 (which are generally or substantially coaxial), while rotation of the screw 214 in the other direction causes the drive component 228 to move proximally along the same axis.

As best shown in FIG. 16A, the linear drive component 228 is operably coupled to the jaws 212A, 212B via the distal prongs 234 such that linear movement of the drive component 228 along the longitudinal axis of the drive component 228 causes the jaws 212A, 212B to move between their open and closed positions. More specifically, each of the jaws 212A, 212B has a proximal body 238A, 238B with a slot 240A, 240B defined therein. Further, as shown in FIG. 16A, a drive pin 242 (similar to pins 80, 82 as discussed above) can be disposed through the opening 236 in the linear drive component 228 and further be positioned within the slots 240A, 240B of the proximal bodies 238A, 238B of the jaws 212A, 212B. As such, linear movement of the linear drive component 228 causes the drive pin 242 to move linearly along the axis of the drive component 228 within the slots 240A, 240B, thereby causing the jaws 212A, 212B to rotate around the axis 220 between the open and closed positions. It is noted that the configuration of the jaw joint 220 can include a pin 220 (that can be substantially similar to pins 80, 82 above) such that the jaws 212A, 212B rotate around the pin 220 that creates the axis 220 as discussed above.

Turning now to the actuation assembly for urging the cutting blade 254 between its retracted and deployed positions (the “blade actuation assembly”), FIGS. 16A-C depict the blade actuation assembly, including a drive or lead screw 250 that is rotatably disposed through a lumen 215 in the jaw actuation drive screw 214. That is, the blade actuation drive screw 250 is disposed within and axially concentric with the jaw actuation drive screw 214. As shown in FIGS. 16A-C, the drive screw 250 has a coupling feature or component 252 at its proximal end that is operably coupleable to a drive motor (not shown) within the forearm (not shown). More specifically, in one embodiment, the coupling component 252 is a torx component 252, but alternatively can be any known component for removable coupling to a drive component and/or motor. In use, a drive component (such as a drive shaft or drive gear) (not shown) in the forearm can be operably coupled to the coupling component 252 such that the drive shaft or gear can drive the rotation of the drive screw 250.

As best shown in FIGS. 16A and 16C, the drive screw 250 is operably coupled to the blade 254 such that rotation of the screw 250 causes linear, axial movement of the blade 254 (between the extended and retracted positions) along the longitudinal axis of the end effector 200. More specifically, as best shown in FIG. 16B, the drive screw 250 has threads 256 defined or otherwise disposed at or near the distal end of the screw 250 that mateably couple with the threads (not shown) defined on the inner surface of the lumen (not shown) in the linear drive component 258. The inner lumen of the linear drive component 258 and the threads therein are substantially similar to those corresponding components on the linear drive component 228 discussed above. For the blade actuation assembly, however, the linear drive component 258 is fixedly attached to or near the proximal end of the blade 254, as best shown in FIGS. 16A and 16C. Alternatively, the linear drive component 258 is integral with a proximal portion of the blade 254. As best shown in FIG. 16C, the threads 256 of the drive screw 250 are positioned within the lumen (not shown) of the linear drive component 258 and mate with the threads (not shown) therein such that rotation of the drive screw 250 causes linear movement of the linear drive component 258. Rotation of the screw 250 in one direction causes the linear drive component 258 (and thus the blade 254) to move distally along the longitudinal axes of the screw 250 and drive component 258 (which are generally or substantially coaxial), while rotation of the screw 250 in the other direction causes the drive component 258 (and blade 254) to move proximally along the same axis.

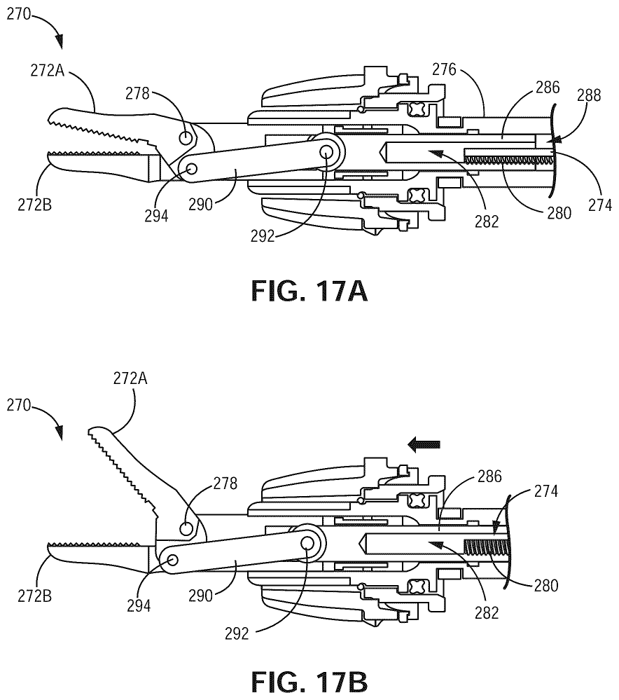

Yet another end effector 270 embodiment is depicted in FIGS. 17A and 17B. In this exemplary version, the end effector 270 has an actuation assembly for urging one (instead of both) of the two jaws 272A, 272B to move between its open and closed positions. As will be described in further detail below, the jaw actuation assembly includes a drive screw 274 rotatably coupled to a linear drive component 284 that is operably coupled to the jaw 272A such that rotation of the drive screw 274 causes axial or linear movement of the linear drive component 284, which causes rotation of the jaw 272A around the jaw joint 278.

As shown in FIGS. 17A-B, the jaw actuation assembly has a drive or lead screw 274 that is rotatably disposed through the end effector body 276. Like the bodies 51, 216 as described above, in certain embodiments, the end effector body 276 can have two coupleable sections (not shown), can be made up of three or more coupleable sections, or can be a single unitary component as shown. In one embodiment, the drive screw 274 can have a coupling feature or component (not shown) at its proximal end similar to the proximal coupling components (such as component 218) discussed above. Alternatively, the proximal end of the drive screw 274 can have any other known coupling component or can be driven by any other known mechanism, component, or feature.

As shown in FIGS. 17A-B, the drive screw 274 is operably coupled to the jaw 272A, such that rotation of the screw 274 causes rotation of the jaw 272A (between the open and closed positions) around a jaw joint 278. More specifically, the drive screw 274 has threads 280 defined or otherwise disposed at or near the distal end of the screw 274 that mateably couple with threads (not shown) defined on the inner surface of the lumen 282 in the linear drive component 284. Much like the linear drive components 66, 228, 258 as discussed above, the linear drive component 284 has a proximal tube 286 with the lumen 282 defined therethrough that is accessible via a proximal opening 288 (as best shown in FIG. 17A). Further, a connecting link 290 is rotatably coupled to or near the distal end of the drive component 284 via a coupling pin 292. The connecting link 290 can be a bar, rod, or any other structure that can couple the linear drive component 284 to the first jaw 272A. That is, the distal end or a distal portion of the connecting link 290 is rotatably coupled to a proximal portion of the jaw 272A via a coupling pin 294.

As shown in FIGS. 17A-B, the threads 280 of the drive screw 274 are positioned through the opening 288 in the proximal end of the proximal tube 286 of the linear drive component 284 and mate with the threads (not shown) on the inner surface of the lumen 282 therein such that rotation of the drive screw 274 causes linear movement of the linear drive component 284. Rotation of the screw 274 in one direction causes the linear drive component 284 to move distally along the longitudinal axes of the screw 274 and drive component 284 (which are generally or substantially coaxial), while rotation of the screw 274 in the other direction causes the drive component 284 to move proximally along the same axis.

As noted above, the linear drive component 284 is operably coupled to the first jaw 272A via the connecting link 290 such that linear movement of the drive component 284 along the longitudinal axis of the drive component 284 causes the first jaw 272A to move between its open and closed positions. More specifically, the proximal end of the jaw 272A has an opening (not shown) through which the coupling pin 294 as discussed above is positioned. As such, linear movement of the linear drive component 284 causes the connecting link 290 to move linearly along the axis of the drive component 284 and causing the proximal end of the jaw 272A to move in a similar fashion, thereby causing the jaw 272A to rotate around the axis 278 between the open and closed positions. It is noted that the configuration of the jaw joint 278 can include a pin 278 (that can be substantially similar to pins 80, 82 above) such that the jaw 272A rotates around the pin 278 that creates the axis 278 as discussed above.

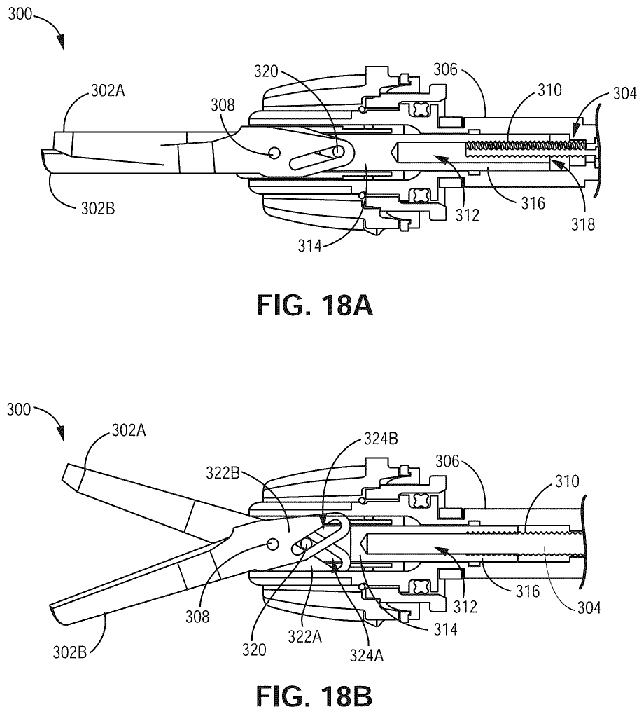

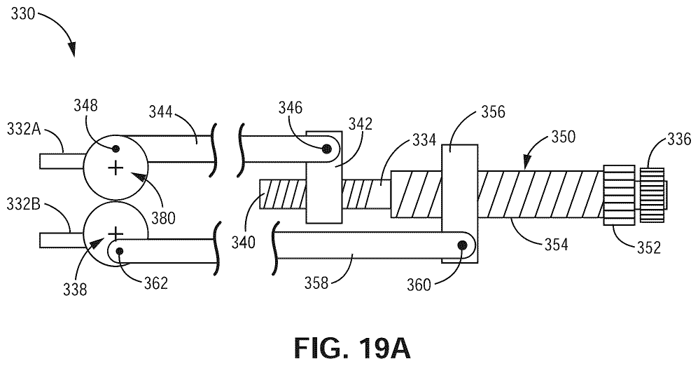

A further end effector 300 embodiment is depicted in FIGS. 18A and 18B. In this exemplary version, the end effector 300 has an actuation assembly for urging both jaws 302A, 302B to move between their open and closed positions. As will be described in further detail below, the jaw actuation assembly includes a drive screw 304 rotatably coupled to a linear drive component 314 that is operably coupled to the jaws 302A, 302B such that rotation of the drive screw 304 causes axial or linear movement of the linear drive component 314, which causes rotation of the jaws 302A, 302B around the jaw joint 308.

As shown in FIGS. 18A-B, the jaw actuation assembly has a drive or lead screw 304 that is rotatably disposed through the end effector body 306. Like the bodies 51, 216 as described above, in certain embodiments, the end effector body 306 can have two coupleable sections (not shown), can be made up of three or more coupleable sections, or can be a single unitary component as shown. In one embodiment, the drive screw 304 can have a coupling feature or component (not shown) at its proximal end similar to the proximal coupling components (such as component 218) discussed above. Alternatively, the proximal end of the drive screw 304 can have any other known coupling component or can be driven by any other known mechanism, component, or feature.

As shown in FIGS. 18A-B, the drive screw 304 is operably coupled to the jaws 302A, 302B such that rotation of the screw 304 causes rotation of the jaws 302A, 302B (between the open and closed positions) around a jaw joint 308. More specifically, the drive screw 304 has threads 310 defined or otherwise disposed at or near the distal end of the screw 304 that mateably couple with threads (not shown) defined on the inner surface of the lumen 312 in the linear drive component 314. Much like the linear drive components 66, 228, 258 as discussed above, the linear drive component 314 has a proximal tube 316 with the lumen 312 defined therethrough that is accessible via a proximal opening 318 (as best shown in FIG. 18A).

Further, the linear drive component 314 has a drive pin 320 (similar to pins 80, 82 as discussed above) that is attached thereto or otherwise associated therewith. In embodiment, the drive pin 320 can be disposed through an opening in the linear drive component in a fashion similar to the pin 80 as discussed above. The drive pin 320 is positioned within the slots 324A, 324B of the proximal bodies 322A, 322B of the jaws 302A, 302B. As such, linear movement of the linear drive component 314 causes the drive pin 320 to move linearly along the axis of the drive component 314 within the slots 324A, 324B, thereby causing both jaws 302A, 302B to rotate around the axis 308 between the open and closed positions.

As shown in FIGS. 18A-B, the threads 310 of the drive screw 304 are positioned through the opening 318 in the proximal end of the proximal tube 316 of the linear drive component 314 and mate with the threads (not shown) on the inner surface of the lumen 312 therein such that rotation of the drive screw 304 causes linear movement of the linear drive component 314. Rotation of the screw 304 in one direction causes the linear drive component 314 to move distally along the longitudinal axes of the screw 304 and drive component 314 (which are generally or substantially coaxial), while rotation of the screw 304 in the other direction causes the drive component 314 to move proximally along the same axis.

As noted above, the linear drive component 314 is operably coupled to the two jaws 302A, 302B via the drive pin 320 such that linear movement of the drive component 314 along the longitudinal axis of the drive component 314 causes the two jaws 302A, 302B to move between their open and closed positions, as described above. As such, linear movement of the linear drive component 314 causes the drive pin 320 to move linearly along the axis of the drive component 314, thereby causing the jaws 302A, 302B to rotate around the axis 308 between the open and closed positions. It is noted that the configuration of the jaw joint 308 can include a pin 308 (that can be substantially similar to pins 80, 82 above) such that the jaws 302A, 302B rotate around the pin 308 that creates the axis 308 as discussed above.

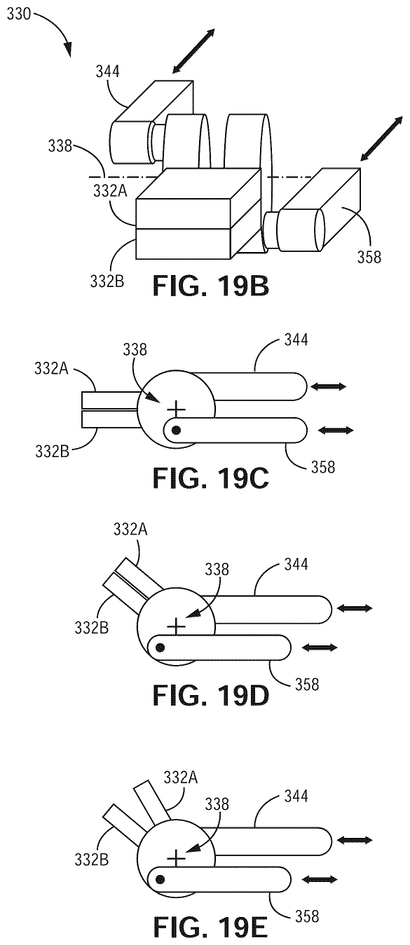

In accordance with a further implementation, yet another exemplary version of an end effector 330 is depicted schematically in FIGS. 19A-19E having a jaw actuation assembly for urging the jaws 332A, 332B between their open and closed positions while also allowing the jaws 332A, 332B to move more freely than other similar embodiments, such that the end effector 330 has a “wrist action” by which both jaws 332A, 332B can be rotated to other rotational positions and still move between their open and closed positions. As will be described in further detail below, the jaw actuation assembly includes separate actuation assemblies for each of the jaws 332A, 332B. That is, the first jaw 332A has a drive screw 334 rotatably coupled to a linear drive component 342 that is operably coupled to the first jaw 332A such that rotation of the drive screw 334 causes axial or linear movement of the linear drive component 342, which causes rotation of the first jaw 332A. Similarly, the second jaw 332B has a drive screw 350 rotatably coupled to a linear drive component 356 that is operably coupled to the second jaw 332B such that rotation of the drive screw 350 causes axial or linear movement of the linear drive component 356, which causes rotation of the second jaw 332B.

Turning first to the actuation assembly for the first jaw 332A (the “first jaw actuation assembly”), FIG. 19A depicts the first jaw actuation assembly, including a drive or lead screw 334 having a coupling feature or component 336 at its proximal end that is operably coupleable to a drive motor (not shown) within the forearm (not shown). More specifically, in one embodiment, the coupling component 336 is a torx component 336, but alternatively can be any known component for removable coupling to a drive component and/or motor. In use, a drive component (such as a drive shaft or drive gear) (not shown) in the forearm can be operably coupled to the coupling component 336 such that the drive shaft or gear can drive the rotation of the drive screw 334.

Continuing with FIG. 19A, the drive screw 334 is operably coupled to the jaw 332A such that rotation of the screw 334 causes rotation of the jaw 332A around a center of rotation 338. More specifically, the drive screw 334 has threads 340 defined or otherwise disposed at or near the distal end of the screw 334 that mateably couple with the threads (not shown) defined on the inner surface of the lumen (not shown) in the linear drive component 342. That is, much like the linear drive components 66, 228, 258, 234 as discussed above, the linear drive component 342 has a lumen (not shown) defined therethrough such that the drive screw 334 is disposed therethrough and the threads 340 of the drive screw 334 mate with the threads (not shown) on the inner surface of the lumen (not shown) therein such that rotation of the drive screw 334 causes linear movement of the linear drive component 342. Rotation of the screw 334 in one direction causes the linear drive component 342 to move distally along the longitudinal axis of the screw 334, while rotation of the screw 334 in the other direction causes the drive component 342 to move proximally along the same axis.

A connecting link 344 is rotatably coupled to or near the distal end of the drive component 342 via a coupling pin 346. The connecting link 344 can be a bar, rod, or any other structure that can couple the linear drive component 342 to the first jaw 332A. That is, the distal end or a distal portion of the connecting link 344 is rotatably coupled to a proximal portion of the jaw 332A via a coupling pin 348. Thus, the linear drive component 342 is operably coupled to the first jaws 332A via the connecting link 344 such that linear movement of the drive component 342 along the longitudinal axis of the drive component 342 causes the jaw 332A to rotate around its center of rotation 338.

Turning now to the actuation assembly for the second jaw 332B (the “second jaw actuation assembly”), FIG. 19A depicts the second jaw actuation assembly, including a drive or lead screw 350 having a coupling feature or component 352 at its proximal end that is operably coupleable to a drive motor (not shown) within the forearm (not shown). More specifically, in one embodiment, the coupling component 352 is a torx component 352, but alternatively can be any known component for removable coupling to a drive component and/or motor. In use, a drive component (such as a drive shaft or drive gear) (not shown) in the forearm can be operably coupled to the coupling component 352 such that the drive shaft or gear can drive the rotation of the drive screw 350.

Continuing with FIG. 19A, the drive screw 350 is operably coupled to the jaw 332B such that rotation of the screw 350 causes rotation of the jaw 332B around a center of rotation 338. More specifically, the drive screw 350 has threads 354 defined or otherwise disposed at or near the distal end of the screw 350 that mateably couple with the threads (not shown) defined on the inner surface of the lumen (not shown) in the linear drive component 356. That is, much like the linear drive components 66, 228, 258, 234 as discussed above, the linear drive component 356 has a lumen (not shown) defined therethrough such that the drive screw 350 is disposed therethrough and the threads 354 of the drive screw 350 mate with the threads (not shown) on the inner surface of the lumen (not shown) therein such that rotation of the drive screw 350 causes linear movement of the linear drive component 356. Rotation of the screw 350 in one direction causes the linear drive component 356 to move distally along the longitudinal axis of the screw 350, while rotation of the screw 350 in the other direction causes the drive component 356 to move proximally along the same axis.

A connecting link 358 is rotatably coupled to or near the distal end of the drive component 356 via a coupling pin 360. The connecting link 358 can be a bar, rod, or any other structure that can couple the linear drive component 356 to the second jaw 332B. That is, the distal end or a distal portion of the connecting link 358 is rotatably coupled to a proximal portion of the jaw 332B via a coupling pin 362. Thus, the linear drive component 356 is operably coupled to the second jaw 332B via the connecting link 358 such that linear movement of the drive component 356 along the longitudinal axis of the drive screw 350 causes the jaw 332B to rotate around its center of rotation 338.

In the embodiment as shown in FIGS. 19B-19E, the jaws 332A, 332B share the same center of rotation 338. In such embodiments (when the jaws 332A, 332B share the same center of rotation 338), the two jaws 332A, 332B of the end effector 330 can move independently. As such, actuating solely the first jaw actuation assembly or the second jaw actuation assembly as described above would cause the end effector 330 to behave as a single acting jaw, with only the jaw coupled to the actuated assembly moving. On the other hand, if both actuation assemblies are actuated, both jaws 332A, 332B move and the end effector 330 behaves as a double acting jaw, with both jaws moving.

The “wrist” action of this embodiment is shown in additional detail in FIGS. 19D and 19E. That is, FIG. 19D depicts the jaws 332A, 332B re-oriented in one direction (e.g. upward) by actuating to two actuation assemblies in opposite directions. That is, the first connecting link 344 is urged proximally, while the second connecting link 358 is urged distally, thereby moving the jaws 332A, 332B as shown.

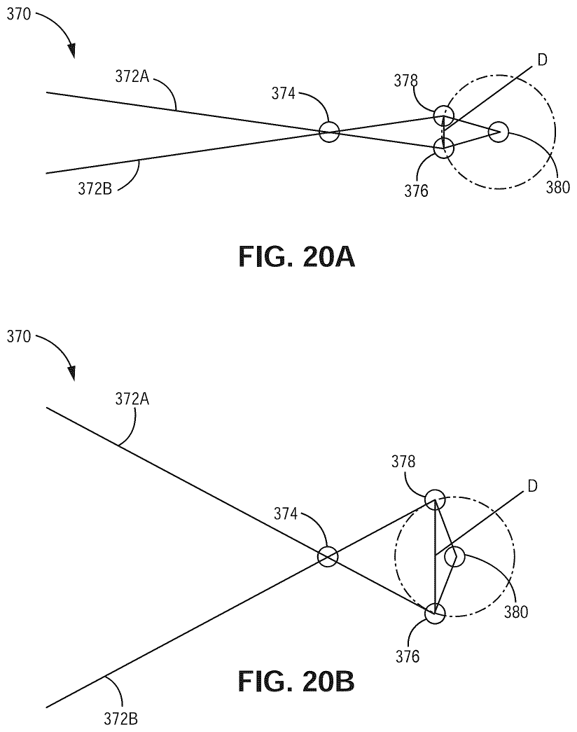

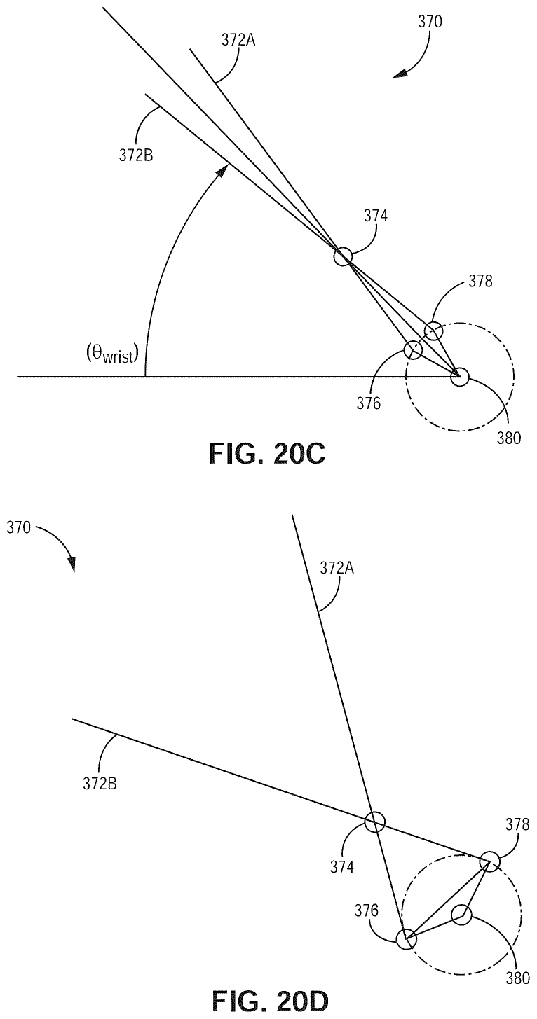

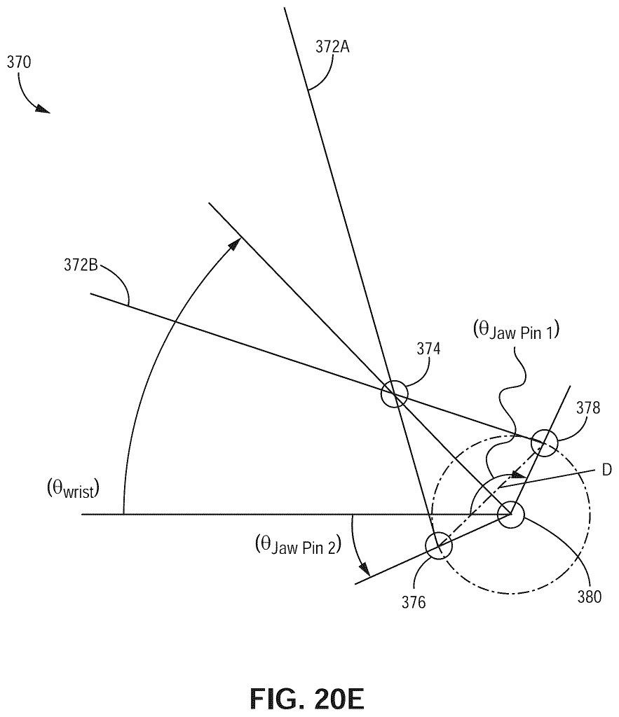

Another possible orientation of the jaws 332A, 332B is shown in FIG. 19E. To achieve this orientation, the actuation assemblies are actuated to move the jaws 332A, 332B apart (away from each other) as in an open/close motion while the two jaws 332A, 332B have an upward orientation as shown. In other words, the configuration of the end effector 330 as shown in FIGS. 19A-19E allows the two jaws 332A, 332B to move between open and closed positions while disposed in various different orientations in relation to the longitudinal axis of the drive screws 334, 350. While standard jaws typically are disposed in a closed configuration such that both jaws are substantially parallel with a longitudinal axis of the end effector body, the independently rotating jaws 332A, 332B of the current embodiment can be disposed in a closed configuration such that the two jaws are in contact with each other but are not substantially parallel with the longitudinal axis of the end effector body. Instead, the two jaws 332A, 332B can be disposed at substantially any angle up to almost 90 degrees in relation to the longitudinal axis of the end effector body. Further, the jaws 332A, 332B can also move into an open configuration while disposed at any of these angles, including as shown in FIG. 19E (and in FIGS. 20C-E as discussed below as well).

In summary, the two separate actuation assemblies along with the shared center of rotation 338 makes it possible for the two jaws 332A, 332B to be moved independently of each other and in any orientation as desired.

It is noted that the actuation assemblies (the first and second drive screws 334, 350 and the first and second linear drive components (or bodies) 342, 356 operably coupled thereto are substantially similar to and interchangeable with the actuation assemblies of the device 210 in FIGS. 16A-16C. Further, it is noted that the two connecting links 344, 358 can also be coupled to any other operational components of any types of end effectors. In other words, either of the first or second connecting links 344, 358 (or both) can be operably coupled to a first jaw, two jaws, a deployable cutting blade or any other operational component. For example, the first or second connecting link 344, 358 (or both) can be operably coupled to the two jaws 164A, 164B of FIGS. 12-J, the first jaw 272A of FIGS. 17A-B, or the two jaws 302A, 302B of FIGS. 18A-B, and the two connecting links 344, 358 can also be operably coupled to the jaws 372A, 372B of FIGS. 20A-E (discussed below) as well.

A further end effector embodiment is depicted schematically in FIGS. 20A-20E. In this implementation, the end effector 370 has two jaws 372A, 372B connected by a common jaw pin 374. As in the previous end effector 330, each jaw 372A, 372B is independently actuated by separate connecting links (not shown) that are connected to the first jaw pin 376 and the second jaw pin 378, wherein the first and second jaw pins 376, 378 rotate about a common center of rotation 380. In FIG. 20A, the jaws 372A, 372B are disposed in a mostly closed configuration. In this end effector embodiment, the open/closed position of the jaws 372A, 372B is determined by the distance between the two jaw pins 376, 378 given by line D as shown in the figure.

Another configuration of the end effector 370 is shown in FIG. 20B, in which the jaws 372A, 372B are in a substantially open configuration in comparison to FIG. 20A. In this configuration, the jaw position is again determined by the distance between the two jaw pins 376, 378 represented by line D.

As with the end effector 330, the jaws 372A, 372B can be oriented in many directions. For example, as shown in FIGS. 20C and 20D, the jaw pins 376, 378 are rotated clockwise such that a common rotation of the end effector is achieved (θ_{wrist}). In this configuration, the common rotation is defined as the angle formed to a line that passes through the center of rotation 380 and the common jaw pin 374.

In this configuration, as shown in FIG. 20E, the open/close position of the jaws 372A, 372B is determined by the line D connecting the first and second jaw pins 376, 378, and the common rotation of the instrument (θ_{wrist}) is related to the two jaw pin angles.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

What is claimed is:

1. A vessel sealer end effector comprising:
(a) an end effector body comprising:
(i) a body lumen defined through the end effector body; and
(ii) a rotatable jaw joint disposed at or near a distal end of the end effector body;
(b) a rotatable jaws drive screw rotatably disposed within the body lumen, the rotatable jaws drive screw comprising:
(i) a proximal screw coupling structure disposed at a proximal end of the rotatable jaws drive screw;
(ii) screw threads disposed around an outer surface of the rotatable jaws drive screw at a distal end of the rotatable jaws drive screw; and
(iii) a drive screw lumen defined within the rotatable jaws drive screw;
(c) first and second jaws rotatably coupled to the end effector body at the rotatable jaw joint, wherein each of the first and second jaws comprise a proximal slot defined in a proximal body of each of the first and second jaws;
(d) a jaws linear drive shaft comprising:
(i) a proximal tubular body comprising a linear drive lumen comprising drive threads disposed on an inner surface of the linear drive lumen, wherein the distal end of the rotatable jaws drive screw is positionable within the linear drive lumen such that the drive threads are mateable with the screw threads;
(ii) distal prongs disposed at a distal end of the jaws linear drive shaft; and
(iii) a drive rod disposed between the distal prongs such that the drive rod is slidably disposed within the proximal slots defined in the first and second jaws; and
(e) a deployable blade slidably disposed through the end effector body, wherein the deployable blade is movable between a retracted position within the end effector body and a deployed position between the first and second jaws.

2. The vessel sealer end effector of claim 1, wherein each of the first and second jaws further comprises:
(a) a structural backbone extending along a length of the jaw, wherein the proximal body extends proximally from the structural backbone;
(b) a first insulation layer disposed around a first portion of the structural backbone;
(c) a second insulation layer disposed around a second portion of the structural backbone;
(d) a contact surface disposed over the first insulation layer and attached to the second insulation layer; and
(e) a blade track formed along a length of the contact surface and the first insulation layer.

3. The vessel sealer end effector of claim 1, wherein the deployable blade comprises:
(a) an elongate proximal rod slidably disposed within the drive screw lumen;
(b) a distal blade body attached to the elongate proximal rod; and
(c) a blade slot defined along a length of the distal blade body, wherein the drive rod is slidably disposed within the blade slot.

4. The vessel sealer end effector of claim 3, further comprising:
(a) an actuation shaft slidably disposed within the drive screw lumen such that the actuation shaft is slidable into contact with a proximal end of the elongate proximal rod; and
(b) a tensioned component operably coupled to the deployable blade, wherein the tensioned component is in an untensioned state when the deployable blade is in the retracted position.

5. The vessel sealer end effector of claim 1, wherein the deployable blade comprises:
(a) a distal blade body;
(b) a blade linear drive shaft disposed near a proximal end of the distal blade body; and
(c) a blade slot defined along a length of the distal blade body, wherein the drive rod is slidably disposed within the blade slot.

6. The vessel sealer end effector of claim 5, wherein the blade linear drive shaft further comprises a linear drive lumen comprising drive threads disposed on an inner surface of the linear drive lumen.

7. The vessel sealer end effector of claim 6, further comprising a rotatable blade drive screw rotatably disposed at least partially within the drive screw lumen, the rotatable jaws drive screw comprising:
(a) a proximal screw coupling structure disposed at a proximal end of the rotatable blade drive screw; and
(b) screw threads disposed around an outer surface of the rotatable blade drive screw at a distal portion of the rotatable blade drive screw, wherein a distal portion of the rotatable blade drive screw is positionable within the linear drive lumen of the blade linear drive shaft such that the drive threads of the linear drive lumen are mateable with the screw threads of the rotatable jaws drive screw.

8. A vessel sealer end effector comprising:
(a) an end effector body comprising:
(i) a body lumen defined through the end effector body; and
(ii) a rotatable jaw joint disposed at or near a distal end of the end effector body;
(b) a rotatable drive screw rotatably disposed within the body lumen, the rotatable drive screw comprising:
(i) a proximal screw coupling structure disposed at a proximal end of the rotatable drive screw;
(ii) screw threads disposed around an outer surface of the rotatable drive screw at a distal end of the rotatable drive screw; and
(iii) a drive screw lumen defined within the rotatable drive screw;
(c) first and second jaws rotatably coupled to the end effector body at the rotatable jaw joint, wherein each of the first and second jaws comprise a proximal slot defined in a proximal body of each of the first and second jaws;
(d) a linear drive shaft comprising:
(i) a proximal tubular body comprising a linear drive lumen comprising drive threads disposed on an inner surface of the linear drive lumen, wherein the distal end of the rotatable drive screw is positionable within the linear drive lumen such that the drive threads are mateable with the screw threads;
(ii) a hub disposed at a distal end of the linear drive shaft; and
(iii) drive tabs disposed on the hub such that the drive tabs are slidably disposed within the proximal slots defined in the first and second jaws; and
(e) a deployable blade slidably disposed through the end effector body, wherein the deployable blade is movable between a retracted position within the end effector body and a deployed position between the first and second jaws.

9. The vessel sealer end effector of claim 8, wherein the hub comprises two channels defined in an outer surface of the hub, wherein the two channels are sized and shaped to receive elongate wires electrically coupled to the first and second jaws.

10. A vessel sealer end effector comprising:
(a) an end effector body comprising a body lumen defined through the end effector body;
(b) a first rotatable drive rod rotatably disposed within the body lumen, the first rotatable drive rod comprising:
(i) a first proximal rod coupling structure disposed at a proximal end of the first rotatable drive rod;
(ii) first rod threads disposed around an outer surface of the first rotatable drive rod along a distal portion of the first rotatable drive rod; and
(iii) a first drive rod lumen defined within the first rotatable drive rod;
(c) a first linear drive body comprising:
(i) a first linear drive lumen defined within the first linear drive body; and
(ii) first drive threads disposed on an inner surface of the first linear drive lumen, wherein a distal portion of the first rotatable drive rod is positionable within the first linear drive lumen such that the first drive threads are mateable with the first rod threads;
(d) a first connecting link operably coupled to the first linear drive body;
(e) a second rotatable drive rod rotatably disposed at least partially within the first drive rod lumen, the second rotatable drive rod comprising:
(i) a second proximal rod coupling structure disposed at a proximal end of the second rotatable drive rod; and
(ii) second rod threads disposed around an outer surface of the second rotatable drive rod along a distal portion of the second rotatable drive rod;
(f) a second linear drive body comprising:
(i) a second linear drive lumen defined within the second linear drive body; and
(ii) second drive threads disposed on an inner surface of the second linear drive lumen,
wherein a distal portion of the second rotatable drive rod is positionable within the second linear drive lumen such that the second drive threads are mateable with the second rod threads; and
(g) a second connecting link operably coupled to the second linear drive body.

11. The vessel sealer end effector of claim 10, further comprising a first operational component operably coupled to the first connecting link and a second operational component operably coupled to the second connecting link.

12. The vessel sealer end effector of claim 11, wherein the first operational component comprises a first jaw and the second operational component comprises a second jaw.

13. The vessel sealer end effector of claim 11, wherein the first operational component comprises at least one jaw and the second operational component comprises a deployable blade.

14. The vessel sealer end effector of claim 13, wherein
(a) the at least one jaw comprises:
(i) a rotatable first jaw comprising a proximal body, wherein the proximal body comprises a slot defined therein; and
(ii) a stationary second jaw; and
(b) the first connecting link comprises a pin, wherein the pin is slidably disposed within the slot,
wherein linear movement of the first connecting link causes rotation of the rotatable first jaw.

15. The vessel sealer end effector of claim 13, wherein
(a) the at least one jaw comprises first and second rotatable jaws, wherein each of the first and second rotatable jaws comprises a proximal body, wherein the proximal body comprises a slot defined therein; and
(b) the first connecting link comprises a pin, wherein the pin is slidably disposed within the slots of the first and second rotatable jaws, wherein linear movement of the first connecting link causes rotation of the first and second jaws.

16. The vessel sealer end effector of claim 10, further comprising:
(a) a first jaw rotatably coupled to the first connecting link at a first center of rotation; and
(b) a second jaw rotatably coupled to the second connecting link at a second center of rotation,
wherein the first and second jaws can rotate independently of each other.

17. The vessel sealer end effector of claim 16, wherein the first and second centers of rotation are coaxial.

18. The vessel sealer end effector of claim 17, wherein the first and second jaws are configurated to be positioned in a closed configuration, wherein the first and second jaws are not parallel with a longitudinal axis of the end effector body.

19. The vessel sealer end effector of claim 18, wherein the first and second jaws are configured to move from the closed configuration into an open configuration.

20. The vessel sealer end effector of claim 10, wherein the second proximal rod coupling structure is disposed proximally of the first proximal rod coupling structure.
